(19) United States
(12) Patent Application Publication
(10) Pub. No.: US 2024/0100027 A1
(43) Pub. Date: Mar. 28, 2024

(54) INHIBITION OF PROSTAGLANDIN DEGRADING ENZYME 15-PGDH TO IMPROVE JOINT STRUCTURE AND FUNCTION

(71) Applicant: The Board of Trustees of the Leland Stanford Junior University, Stanford, CA (US)

(72) Inventors: Helen M. BLAU, Stanford, CA (US); Nidhi BHUTANI, Stanford, CA (US); Mamta SINGLA, Stanford, CA (US); Adelaida Rosa PALLA, Stanford, CA (US)

(73) Assignee: The Board of Trustees of the Leland Stanford Junior University, Stanford, CA (US)

(21) Appl. No.: 18/254,441

(22) PCT Filed: Dec. 9, 2021
(86) PCT No.: PCT/US2021/072843

§ 371 Date: May 25, 2023

Related U.S. Application Data
(60) Provisional application No. 63/123,061, filed on Dec. 9, 2020.

(51) Int. Cl. A61K 31/4365; A61K 31/7105; A61P 19/02

(52) U.S. Cl. CPC A61K 31/4365; A61K 31/7105; A61P 19/02

(57) ABSTRACT

The present disclosure provides methods of improving the structure and/or function of a joint tissue of a subject by administering to the subject an amount of a 15-PGDH inhibitor effective to inhibit 15-PGDH activity and/or reduce 15-PGDH levels in the subject. The methods described herein are useful for treating joint dysfunction and/or degeneration associated with aging, injury, disease, disorder, and/or condition.

### CROSS-REFERENCE

This application claims priority to U.S. Provisional Patent Application No. 63/123,061, filed Dec. 9, 2020, the disclosure of which is herein incorporated by reference in its entirety.

### STATEMENT AS TO RIGHTS TO INVENTIONS MADE UNDER FEDERALLY SPONSORED RESEARCH AND DEVELOPMENT

This invention was made with Government support under contract AG020961 awarded by the National Institutes of Health. The Government has certain rights in the invention.

### BACKGROUND

Dysfunction and/or degeneration of tissues of the joint can have a significant detrimental impact on quality of life and normal functioning. Joint tissue dysfunction and/or degeneration can be a result of aging, injury, disease, disorder, condition, or other unexplained causes. For example, osteoarthritis is a chronic disease of the joints that is increasing in incidence and is a leading cause of disability affecting millions of people in the United States and worldwide. However, no disease-modifying drug is available for treatment of osteoarthritis. Osteoarthritis and other conditions affecting the joint can affect various joint tissues in a variety of ways, leading to negative impacts such as reduced mobility, soreness, chronic pain, etc.

As with osteoarthritis, treatment options for joint tissue dysfunction and/or degeneration in general are limited. Thus, there remains a need in the art for effective strategies to improve joint tissue structure and/or function, thereby improving quality of life. The present disclosure provides methods to satisfy this need in addition to other advantages.

### BRIEF SUMMARY

The present disclosure provides methods of improving the structure and/or function of a joint tissue of a subject by administering to the subject an amount of a 15-hydroxyprostaglandin dehydrogenase (15-PGDH) inhibitor effective to inhibit 15-PGDH activity and/or reduce 15-PGDH levels in the subject. In some embodiments, the methods comprise improving the structure of a joint tissue of a subject. In some embodiments, the methods comprise improving the function of a joint tissue of a subject. In some embodiments, the administering increases a level of prostaglandin E2 (PGE2) and/or prostaglandin D2(PGD2) in the joint tissue of the subject. In some embodiments, a level of PGE2 and/or PGD2 in the joint tissue is increased relative to the joint tissue prior to the administering of the 15-PGDH inhibitor. In some embodiments, a level of PGE2 and/or PGD2 in the joint tissue is increased (e.g., by at least about 10%) relative to the joint tissue prior to the administering of the 15-PGDH inhibitor.

In some embodiments of the methods described herein, the joint tissue of the subject displays at least one marker of dysfunction and/or degeneration. In some embodiments, the at least one marker is selected from the group consisting of decreased GAG staining, reduction in cartilage thickness, increased fibrillation, reduction in cartilage surface smoothness, reduction in bone tissue density, increased OARSI score, decreased levels of sGAG, decreased cell proliferation, increased pain and/or pain related behaviors in the subject, increased expression of Indian hedgehog (Ihh) protein, increased expression of catabolic genes (e.g., matrix metalloprotease (MMP)-13, MMP-3, MMP-1, aggrecanase, a disintegrin and metalloproteinase with thrombospondin motifs 4 (ADAMTS4), ADAMTS5, cathepsins, or combinations thereof), decreased expression of anabolic chondrocyte genes (e.g., Sox9, collagen II, Acan, or combinations thereof), decreased expression of genes involved in mitochondrial function and metabolism, increased expression of genes involved in mitochondrial dysfunction and/or osteoarthritis, decreased mitochondrial biogenesis, decreased mitochondrial levels, increased levels of type X collagen, increased chondrocyte size, increased chondrocyte sphericity, increased levels of cytokines and/or chemokines (e.g., IL1, IL6, IL15, oncostatin M, tumor necrosis factor, or combinations thereof), increased levels of inflammatory mediators (e.g., nitric oxide, reactive oxygen species, complement activation, or combinations thereof), increased levels of cell-derived and/or matrix-derived products (e.g., alarmins such as S100, fibronectin fragments, hyaluronic acid fragments, collagen fragments, proteoglycan fragments, high-mobility group box, or combinations thereof), and a combination thereof. In some embodiments, the administering reduces the at least one marker of joint tissue dysfunction and/or degeneration. In some embodiments, the joint tissue dysfunction and/or degeneration in the subject is a result of aging. In some embodiments, the joint tissue dysfunction and/or degeneration in the subject is a result of injury (e.g., cartilage injury, joint injury, trauma, anterior cruciate ligament (ACL) tear, meniscus tear, hip labral tear, rotator cuff injury, spondylosis, spinal fractures, hip fractures, degenerative spondylolisthesis, slipped disc, herniated disc, and combinations thereof). In some embodiments, the joint tissue dysfunction and/or degeneration in the subject is a result of disease, disorder, or condition (e.g., osteoarthritis, other arthritis types, osteoporosis, rheumatoid arthritis, juvenile idiopathic arthritis, gout, systemic lupus erythematosus, seronegative spondyloarthropathy, degenerative disc disease, congenital cartilage disorders, bone disorders, and combinations thereof). In some embodiments, the disease, disorder, or condition is osteoarthritis.

In some embodiments of the methods described herein, a level of PGE2 and/or PGD2 in the joint tissue is increased to a level substantially similar to a level present in a joint tissue of a subject not displaying the at least one marker of dysfunction and/or degeneration. In some embodiments, a level of PGE2 and/or PGD2 in the joint tissue is increased to a level within 10% to 200% of a level present in a joint tissue of a subject not displaying the at least one marker of dysfunction and/or degeneration. In some embodiments, the joint tissue is selected from the group consisting of cartilage, synovium, bone, bone marrow, ligament, tendon, bursa, meniscus, and combinations thereof.

In some embodiments of the methods described herein, the structure and/or function of the joint tissue is improved relative to the joint tissue prior to the administering of the 15-PGDH inhibitor. In some embodiments, the method results in increased GAG staining, increased cartilage thickness, decreased fibrillation, increased cartilage surface smoothness, increased bone tissue density, decreased OARSI score, increased levels of sGAG, increased cell proliferation, decreased pain and/or pain-related behaviors in the subject, decreased expression of Indian hedgehog (Ihh) protein, decreased expression of catabolic genes (e.g., MMP-13, MMP-3, MMP-1, aggrecanase, ADAMTS4, ADAMTS5, cathepsins, or combinations thereof), increased expression of anabolic chondrocyte genes (e.g., Sox9, collagen II, Acan, or combinations thereof), increased expression of genes involved in mitochondrial function and metabolism, decreased expression of genes involved in mitochondrial dysfunction and/or osteoarthritis, increased mitochondrial biogenesis, increased mitochondria levels, decreased levels of type X collagen, decreased chondrocyte size, decreased chondrocyte sphericity, decreased levels of cytokines and/or chemokines (e.g., IL 1, IL6, IL15, oncostatin M, tumor necrosis factor, or combinations thereof), decreased levels of inflammatory mediators (e.g., nitric oxide, reactive oxygen species, complement activation, or combinations thereof), decreased levels of cell-derived and/or matrix-derived products (e.g., alarmins such as S100, fibronectin fragments, hyaluronic acid fragments, collagen fragments, proteoglycan fragments, high-mobility group box, or combinations thereof), or any combination thereof.

In some embodiments, the methods described herein result in a decreased level of a PGE2 and/or PGD2 metabolite in the joint tissue relative to the joint tissue prior to the administering of the 15-PGDH inhibitor. In some embodiments, the methods result in a level of a PGE2 and/or PGD2 metabolite in the joint tissue that is substantially similar to a level present in a joint tissue of a subject not displaying the at least one marker of dysfunction and/or degeneration. In some embodiments, the PGE2 and/or PGD2 metabolite is selected from the group consisting of 15-keto PGE2 and 13,14-dihydro-15-keto PGE2.

In some embodiments of the methods described herein, the 15-PGDH inhibitor is selected from the group consisting of a small molecule compound, a blocking antibody, a nanobody, and a peptide. In some embodiments, the 15-PGDH inhibitor is SW033291. In some embodiments, the 15-PGDH inhibitor is selected from the group consisting of an antisense oligonucleotide, microRNA, siRNA, and shRNA.

In some embodiments of the methods described herein, the subject is a human. In some embodiments, the subject is less than 30 years of age. In some embodiments, the subject is at least 30 years of age. In some embodiments, the 15-PGDH inhibitor reduces or blocks 15-PGDH expression. In some embodiments, the 15-PGDH inhibitor reduces or blocks enzymatic activity of 15-PGDH.

Other objects, features, and advantages of the present disclosure will be apparent to one of skill in the art from the following detailed description and figures.

### BRIEF DESCRIPTION OF THE DRAWINGS

FIG. 1 shows that systemic inhibition of 15-PGDH with a small molecule regenerates cartilage in aged mice, according to aspects of this disclosure. The top panel shows an experimental scheme for systemic inhibition of 15-PGDH in 24-month-old mice treated intraperitoneally (i.p.) with vehicle or a small molecule inhibitor of 15-PGDH, SW0033291 (SW)every day for a month. Mice were evaluated after one month. The bottom left panel shows representative images of Safranin O (Saf O)/fast green staining of knee joints in aged mice after systemic vehicle (“Aged VEH”) or SW treatment (“Aged SW”). The bottom right panel shows maximum scores (“MAX Score”) and summit scores for the mice knee joints in vehicle or SW treated mice based on the OARSI scoring system.

FIG. 2 shows that local 15-PGDH inhibition in the knee by intra-articular SW injections attenuates post-traumatic osteoarthritis (OA) in adult mice, according to aspects of this disclosure. The top left panel shows an experimental scheme for tibia loading for induction of OA in 3-month-old adult mice. Intra-articular injections of vehicle or SW were given twice a week for 4 weeks and joints were evaluated at 6 weeks after tibia loading. The top right panel shows representative images of Safranin O (SafD)/fast green staining of OA knee joints treated locally with vehicle or SW. The bottom left panel shows maximum scores (“MAX Score”) and summit scores for the mice knee joints in vehicle or SW treated mice based on the OARSI scoring system. The bottom right panel shows fold change in significantly altered cytokines obtained from a 48-plex Luminex assay between vehicle and SW treated groups. Welch t-test applied for statistical significance, * denotes P<0.05, ***denotes P<0.001, and ****denotes P<0.0001.

FIG. 3 shows that 15-PGDH inhibition alters multiple pathways to rejuvenate cartilage in aged mice, according to aspects of this disclosure. The heatmap depicts differentially expressed genes identified from RNA sequencing and analyses in cartilage isolated from young mice (3 months) or aged mice (24 months) treated systemically with vehicle (“Aged Veh”) or SW (“Aged SW”).

FIG. 4 shows that 15-PGDH inhibition boosts mitochondrial biogenesis in aged cartilage, according to aspects of this disclosure. The top panel shows representative TEM images of chondrocytes in articular cartilage of mice joints in untreated young mice and aged mice treated with vehicle (“Aged Veh”) or SW (“Aged SW”) systemically. For each mouse category, the upper panel shows magnified images of mitochondria in all groups marked by the box in the lower panel. Scale bar=2 uM The bottom panel shows quantification of mitochondria per cell using Image J in the categories described above. One-way ANOVA followed by multiple comparison test was used to determine statistical significance.

FIG. 5 shows that 15-PGDH inhibition in Human OA cartilage explants induces a regenerative response, according to aspects of this disclosure. The top left panel shows representative images of Safranin O (SafD)/fast green staining of human OA cartilage explants (n>5) treated with vehicle (“Control”) or SW (10 uM) for 7 days. The top right panel shows quantification of sulphated glycosaminoglycans (sGAG) by 1,9-dimethylmethylene blue (DMMB) assay in OA cartilage explants (n=5) treated with vehicle (“CTRL”) or SW (10 uM). Paired t-test was applied to determine statistical significance. The bottom panel shows immunofluorescence images of Ki-67 staining of OA cartilage explants from 3 different donors treated with vehicle (“CTRL”) or SW.

FIG. 6 shows that 15-PGDH inhibition in human OA cartilage explants induces a regenerative response, according to aspects of this disclosure. The top panel shows an intensity distribution quantification of Ki-67 staining of OA cartilage explants in vehicle and SW treated groups. The bottom panel shows fold change in significantly altered cytokines obtained from 80-plex Luminex assay between vehicle (“Veh”) and SW treated OA explants from 3 different OA patients. Welch t-test was applied to determine statistical significance, * denotes P<0.05, *** denotes P<0.001, and **** denotes P<0.0001.

### DETAILED DESCRIPTION

### 1. Introduction

Osteoarthritis (OA) is a chronic disease of the joints that is increasing in incidence and is a leading cause of disability affections millions of people in US and worldwide (Litwic et al., 2013. Br. Med. Bull. 105:185-199). No disease-modifying OA drug is available. Aging and trauma to articular cartilage as well as chronic inflammation in the joint are molecular events associated with OA. Development of OA is multifactorial and is considered a disease of the whole joint, affecting cartilage, synovium, and bone, and this disease is affected by age, sex and metabolic status besides genetic susceptibility.

Prostaglandin E2 (PGE2), also known as dinoprostone, has been employed in various clinical settings, including to induce labor in women and to augment hematopoietic stem cell transplantation. PGE2 can be used as an anticoagulant and antithrombotic agent. The role of PGE2 as a lipid mediator that can resolve inflammation is also well known. Nonsteroidal anti-inflammatory drugs (NSAIDs), inhibitors of cyclooxygenase 1 (COX-1) and/or cyclooxygenase 2 (COX-2), suppress inflammation by inhibiting prostanoids, mainly via PGE2 biosynthesis. Prostaglandin D2 (PGD2) is a structural isomer of PGE2, with the 9-keto and 11-hydroxy group on PGE2 reversed on PGD2. PGD2 plays a role in a number of biological functions including vasoconstriction, inflammation, regulation of body temperature during sleep, chemotaxis, and male sexual development. PGE2 and PGD2 are both synthesized from arachidonic acid by cyclooxygenases (COX) and by prostaglandin E synthase enzymes or prostaglandin D synthase enzymes, respectively. Levels of PGE2 and PGD2 are physiologically regulated by the enzyme 15-hydroxyprostaglandin dehydrogenase (15-PGDH), which catalyzes the conversion of the 15-OH group of both PGE2 and PGD2 to a 15-keto group.

The present disclosure is based, in part, on the discovery that inhibition of 15-PGDH inhibits degeneration of joint tissue (e.g., cartilage), and improves tissue structure and function. As such, the methods described herein are useful for improving the structure and/or function of a joint tissue of a subject. Without being bound by the following theory, it is believed that elevated 15-PGDH levels in joint tissues displaying at least one marker of dysfunction and/or degeneration lead to PGE2 and/or PGD2 degradation in these tissues and thus to lower levels of PGE2 and/or PGD2 and of PGE2 and/or PGD2 signaling, which has deleterious effects on joint tissue function. Inhibiting 15-PGDH in joint tissues may restore or increase PGE2 and/or PGD2 levels in these tissues to improve joint tissue structure and/or function.

The data described in the present disclosure provide pre-clinical evidence in three distinct lines of approach: (a) systemic 15-PGDH inhibition by treatment with a small molecule inhibitor regenerates cartilage in aged mice; (b) local 15-PGDH inhibition in the knee by intra-articular small molecule inhibitor treatment attenuates OA in adult mice; and (c)15-PGDH inhibition in OA patient cartilage explants induces a regenerative response. As such, the methods provided herein to inhibit 15-PGDH activity and/or reduce 15-PGDH levels in tissues can potentially improve the structure and/or function of a joint tissue of a subject, slow or reverse joint damage (e.g., in OA), thereby improving patient quality of life and/or outcomes for subjects with joint tissue displaying dysfunction and/or degeneration as a result of aging, injury, disease, disorder, or condition.

### 2. General

Practicing the methods disclosed herein utilizes routine techniques in the field of molecular biology. Basic texts disclosing the general methods of use described herein include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized. e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a.” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The terms “prostaglandin E2”, “PGE2”, and “dinoprostone” refer to prostaglandin that can be synthesized from arachidonic acid via cyclooxygenase (COX) enzymes and terminal prostaglandin E synthases (PGES). PGE2 plays a role in a number of biological functions including vasodilation, inflammation, and modulation of sleep/wake cycles. Structural and functional information about PGE2 can be found, e.g., in the entry for “Dinoprostone” of PubChem: pubchem.ncbi.nlm.nih.gov/compound/Dinoprostone, the contents of which are herein incorporated by reference in their entirety.

The term “prostaglandin D2” or “PGD2” refers to prostaglandin that can be synthesized from arachidonic acid via cyclooxygenase (COX) enzymes and PGD2 synthases (PTDS). PGD2 is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. Structural and functional information about PGD2 can be found, e.g., in the entry for “Prostaglandin D2” of PubChem: pubchem.ncbi.nlm.nih.gov/compound/448457, the contents of which are herein incorporated by reference in their entirety.

“15-PGDH” (15-hydroxyprostaglandin dehydrogenase) is an enzyme involved in the inactivation of a number of active prostaglandins, e.g., by catalyzing oxidation of PGE2 to 15-keto-prostaglandin E2 (15-keto-PGE2), or the oxidation of PGD2 to 15-keto-prostaglandin D2 (15-keto-PGD2). The human enzyme is encoded by the HPGD gene (Gene ID: 3248). The enzyme is a member of the short-chain nonmetalloenzyme alcohol dehydrogenase protein family. Multiple isoforms of the enzyme exist, e.g., in humans, any of which can be targeted using the present methods. For example, any of human isoforms 1-6 (e.g., GenBank Accession Nos. NP_000851.2, NP_001139288.1. NP_001243236.1, NP_001243234.1, NP_001243235.1, NP_001350503.1, NP_001243230.1) can be targeted, as can any isoform with 50%, 60%, 70%, 80%, 85%, 90%, 95%, or higher identity to the amino acid sequences of any of GenBank Accession Nos. NP_000851.2, NP_001139288.1, NP_001243236.1, NP_001243234.1, NP_001243235.1, NP_001350503.1, NP_001243230.1, or of any other 15-PGDH enzyme.

A “15-PGDH inhibitor” refers to any agent that is capable of inhibiting, reducing, decreasing, attenuating, abolishing, eliminating, slowing, and/or counteracting in any way any aspect of the expression, stability, and/or activity of 15-PGDH. A 15-PGDH inhibitor can, for example, reduce any aspect of the expression, e.g., transcription, RNA processing, RNA stability, and/or translation of a gene encoding 15-PGDH, e.g., the human HPGD gene, by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Similarly, a 15-PGDH inhibitor can, for example, reduce the activity. e.g., enzymatic activity, of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Further, a 15-PGDH inhibitor can, for example, reduce the stability of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. A “15-PGDH inhibitor”, also referred to herein as a “15-PGDH agent” or a “15-PGDH compound,” can be any molecule, either naturally occurring or synthetic, e.g., peptide, protein, oligopeptide (e.g., from about 5 to about 25 amino acids in length, e.g., about 5, about 10, about 15, about 20, or about 25 amino acids in length), small molecule (e.g., an organic molecule having a molecular weight of less than about 2500 daltons, e.g., less than about 2000, less than about 1000, or less than about 500 daltons), antibody, nanobody, polysaccharide, lipid, fatty acid, inhibitory RNA (e.g., siRNA, shRNA, microRNA), modified RNA, polynucleotide, oligonucleotide, e.g., antisense oligonucleotide, aptamer, affimer, drug compound, or other compound.

The terms “expression” and “expressed” refer to the production of a transcriptional and/or translational product, e.g., of a nucleic acid sequence encoding a protein (e.g., 15-PGDH). In some embodiments, the term refers to the production of a transcriptional and/or translational product encoded by a gene (e.g., the human HPGD gene) or a portion thereof. The level of expression of a DNA molecule in a cell may be assessed on the basis of either the amount of corresponding mRNA that is present within the cell or the amount of protein encoded by that DNA produced by the cell.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term includes antibody fragments having the same antigen specificity, and fusion products thereof.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “VH” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

The terms “antigen-binding portion” and “antigen-binding fragment” are used interchangeably herein and refer to one or more fragments of an antibody that retains the ability to specifically bind to an antigen (e.g., a 15-PGDH protein). Examples of antibody-binding fragments include, but are not limited to, a Fab fragment (a monovalent fragment consisting of the VL, VH, CL, and CHI domains). F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), VL (light chain variable region), VH (heavy chain variable region), nanobodies, and any combination of those or any other functional portion of an immunoglobulin peptide capable of binding to target antigen (see. e.g., Fundamental Immunology (Paul ed., 4th ed. 2001).

The phrase “specifically binds” refers to a molecule (e.g., a 15-PGDH inhibitor such as a small molecule or antibody) that binds to a target with greater affinity, avidity, more readily, and/or with greater duration to that target in a sample than it binds to a non-target compound. In some embodiments, a molecule that specifically binds a target (e.g., 15-PGDH) binds to the target with at least 2-fold greater affinity than non-target compounds, e.g., at least 3-fold, at least 4-fold, at least 5-fold, at least 6-fold, at least 7-fold, at least 8-fold, at least 9-fold, at least 10-fold, at least 20-fold, at least 25-fold, at least 50-fold or greater affinity. For example, in some embodiments, a molecule that specifically binds to 15-PGDH typically binds to 15-PGDH with at least a 2-fold greater affinity than to a non-15-PGDH target.

The term “derivative,” in the context of a compound, includes but is not limited to, amide, ether, ester, amino, carboxyl, acetyl, and/or alcohol derivatives of a given compound.

The term “treating” or “treatment” refers to any one of the following: ameliorating one or more symptoms of a disease, disorder, or condition; preventing the manifestation of such symptoms before they occur; slowing down or completely preventing the progression of the disease, disorder, or condition (as may be evident by longer periods between reoccurrence episodes, slowing down or prevention of the deterioration of symptoms, etc.); enhancing the onset of a remission period: slowing down the irreversible damage caused in the progressive-chronic stage of the disease, disorder, or condition (both in the primary and secondary stages): delaying the onset of said progressive stage: or any combination thereof.

The term “administer,” “administering.” or “administration” refers to the methods that may be used to enable delivery of agents or compositions such as the compounds described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal injection, oral administration, administration as a suppository, and topical administration. In some cases, the administration is systemic administration (e.g., administration into the circulatory system such that multiple tissues and/or organs are treated or affected). In some cases, the administration is local administration (e.g., directly to the joint tissue or organ such that the joint tissue and/or organ is treated or affected). One skilled in the art will know of additional methods for administering a therapeutically effective amount of the compounds described herein.

The term “therapeutically effective amount” or “therapeutically effective dose” or “effective amount” refers to an amount of a compound (e.g., 15-PGDH inhibitor) that is sufficient to bring about a beneficial or desired clinical effect. A therapeutically effective amount or dose may be based on factors individual to each patient, including, but not limited to, the patient's age, size, type or extent of disease, disorder, or condition, stage of the disease, disorder, or condition, route of administration, the type or extent of supplemental therapy used, and/or ongoing disease process and/or type of treatment desired (e.g., aggressive vs. conventional treatment). Therapeutically effective amounts of a compound or composition (e.g., a 15-PGDH inhibitor), as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “pharmaceutical composition” as used herein refers to a composition comprising a compound (e.g., a 15-PGDH inhibitor) as described herein and one or more pharmaceutically acceptable carriers and/or pharmaceutically acceptable excipients.

The term “pharmaceutically acceptable carrier” as used herein refers to a carrier or a diluent that does not cause significant irritation to an organism and does not abrogate the biological activity and properties of the administered compound.

The terms “subject,” “individual,” and “patient” are used interchangeably herein to refer to a vertebrate, preferably a mammal, more preferably a human. Mammals include, but are not limited to, murines, rats, simians, humans, farm animals or livestock for human consumption such as pigs, cattle, and ovines, as well as sport animals and pets. Subjects also include vertebrates such as fish and poultry.

The term “acute regimen”, in the context of administration of a compound, refers to a temporary or brief application of a compound to a subject, e.g., human subject, or to a repeated application of a compound to a subject, e.g., human subject, wherein a desired period of time (e.g., 1 day) lapses between applications. In some embodiments, an acute regimen includes an acute exposure (e.g., a single dose) of a compound to a subject over the course of treatment or over an extended period of time. In other embodiments, an acute regimen includes intermittent exposure (e.g., repeated doses) of a compound to a subject in which a desired period of time lapses between each exposure.

The term “chronic regimen,” in the context of administration of a compound, refers to a repeated, chronic application of a compound to a subject, e.g., human subject, over an extended period of time such that the amount or level of the compound is substantially constant over a selected time period. In some embodiments, a chronic regimen includes a continuous exposure of a compound to a subject over an extended period of time.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. In particular embodiments, modified RNA molecules are used, e.g., mRNA with certain chemical modifications to allow increased stability and/or translation when introduced into cells, as described in more detail below. It will be appreciated that any of the RNAs used in the present methods, including nucleic acid inhibitors such as siRNA or shRNA, can be used with chemical modifications to enhance, e.g., stability and/or potency. e.g., as described in Dar et al., Scientific Reports 6: article no. 20031 (2016), and as presented in the database accessible at crdd.osdd.net/servers/sirnamod/.

“Polypeptide”, “peptide”, and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity”, in the context of describing two or more polynucleotide or amino acid sequences, refer to two or more sequences or specified subsequences that are the same. Two sequences that are “substantially identical” have at least about 60% identity, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters are used.

### 4. Methods of Improving Joint Structure and/or Function

In one aspect, provided herein is a method for improving the structure and/or function of a joint tissue of a subject, the method comprising: administering to the subject an amount of a 15-PGDH inhibitor effective to inhibit 15-PGDH activity and/or reduce 15-PGDH levels in the subject, thereby improving the structure and/or function of the joint tissue of the subject. The administration of the 15-PGDH inhibitor can be systemic or local, and can enhance any of a number of aspects of the joint tissue including reducing pain and/or pain related behaviors in the subject, enhancing function, physiological activity, endurance, performance on any assay for assessing tissue function; or an improvement in any other measure of joint tissue function or health in the subject (e.g., as described herein).

In some embodiments, the joint tissue is cartilage, synovium, bone, bone marrow, ligament, tendon, bursa, meniscus, and combinations thereof. In some embodiments, the joint tissue is from any joint of a subject (e.g., knee, ankle, hip, hand, wrist, elbow, spinal column, neck, shoulder, etc.).

In some embodiments, the level of PGE2 and/or PGD2 present within the joint tissue may be increased (e.g., after treatment with a 15-PGDH inhibitor, e.g., according to the methods provided herein) relative to the level present in the joint tissue prior to the treatment (e.g., with the 15-PGDH inhibitor) or relative to the level present in a healthy joint tissue from the same subject. The PGE2 and/or PGD2 level in the joint tissue may be increased (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to the level present in the joint tissue prior to the treatment (e.g., with the 15-PGDH inhibitor) or relative to the level present in a healthy joint tissue from the same subject. In some embodiments, the PGE2 and/or PGD2 level in the joint tissue is increased by at least 10%. In some embodiments, the PGE2 and/or PGD2 level in the joint tissue is increased by at least 50%. In some embodiments, the joint tissue of the subject displays at least one marker of dysfunction and/or degeneration and the level of PGE2 and/or PGD2 present in the joint tissue may be increased (e.g., after treatment with a 15-PGDH inhibitor. e.g., according to methods provided herein) to a level substantially similar to a level present in a joint tissue of a subject not displaying the at least one marker of dysfunction and/or degeneration. The PGE2 and/or PGD2 level in the joint tissue may be increased (e.g., by any method disclosed herein) to a level within about 10% to about 200% of a level present in a joint tissue of a subject not displaying the at least one marker of dysfunction and/or degeneration (e.g., about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200% or more). In some embodiments, the PGE2 and/or PGD2 level in the joint tissue is increased to a level within 50% of a level present in a joint tissue of a subject not displaying the at least one marker of dysfunction and/or degeneration.

In some embodiments, the level of a PGE2 and/or PGD2 metabolite present within the joint tissue may be decreased (e.g., after treatment with a 15-PGDH inhibitor, e.g., according to methods provided herein) relative to the level present in the joint tissue prior to the treatment (e.g., with the 15-PGDH inhibitor) or relative to the level present in a healthy joint tissue from the same subject. The PGE2 and/or PGD2 metabolite level in the joint tissue may be decreased (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to the level present in the joint tissue prior to the treatment (e.g., with the 15-PGDH inhibitor) or relative to the level present in a healthy joint tissue from the same subject. In some embodiments, the joint tissue of the subject displays at least one marker of dysfunction and/or degeneration and the level of a PGE2 and/or PGD2 metabolite present within the joint tissue may be decreased (e.g., after treatment with a 15-PGDH inhibitor, e.g., according to methods provided herein) to a level substantially similar to a level present in a joint tissue of a subject not displaying the at least one marker of dysfunction and/or degeneration. The PGE2 and/or PGD2 metabolite level in the joint tissue may be decreased (e.g., by any method disclosed herein) to a level within about 50% or less of a level present in a joint tissue of a subject not displaying the at least one marker of dysfunction and/or degeneration (e.g., within about 40%, within about 35%, within about 30%, within about 25%, within about 20%, within about 15%, within about 10%, within about 5%, or within about 1%). The PGE2 and/or PGD2 metabolite may be 15-keto PGE2, 13,14-dihydro-15-keto PGE2, or both.

In some embodiments, the methods provided herein (e.g., treatment with a 15-PGDH inhibitor) result in improved structure and/or function of a joint tissue of a subject as demonstrated by a decrease in at least one marker of joint tissue dysfunction and/or degeneration. Markers of joint tissue dysfunction and/or degeneration include, but are not limited to, decreased cartilage proteoglycan (GAG) staining, reduction in cartilage thickness, increased fibrillation, reduction in cartilage surface smoothness, reduction in bone tissue density, increased OARSI score (see, e.g., Glasson, et al., 2010, “The OARSI histopathology initiative—recommendations for histological assessments of osteoarthritis in the mouse,” Osteoarthr. Cartil. 18 (Suppl, 3): S17-23), and a combination thereof. Markers of joint tissue dysfunction and/or degeneration may also include, but are not limited to, decreased levels of sulphated glycosaminoglycans (sGAG), decreased cell proliferation (e.g., as measured by a decrease in cells expressing the cell cycle marker Ki67), increased pain and/or pain-related behaviors in the subject, increased expression of Indian hedgehog (Ihh) protein, increased expression of catabolic genes (e.g., matrix metalloprotease (MMP)-13, MMP-3, MMP-1, aggrecanase, a disintegrin and metalloproteinase with thrombospondin motifs 4 (ADAMTS4). ADAMTS5, cathepsins, etc.), decreased expression of anabolic chondrocyte genes (e.g., Sox9, collagen II, Acan, etc.), decreased expression of genes involved in mitochondrial function and metabolism (e.g., Cox5a, Ndufa9), increased expression of genes involved in mitochondrial dysfunction and/or osteoarthritis (e.g., Htra1, Den), decreased mitochondrial biogenesis, decreased mitochondria levels (e.g., mitochondria per cell), increased levels of type X collagen, increased chondrocyte size, increased chondrocyte sphericity, increased levels of cytokines and/or chemokines (e.g., IL1, IL6, IL15, oncostatin M, tumor necrosis factor, CCL7, CXCL10. CCL4, VEGF, IL27, IL2, GMCSF, CCL5, etc.), increased levels of inflammatory mediators (e.g., nitric oxide, reactive oxygen species, complement activation, etc.), increased levels of cell-derived and/or matrix-derived products (e.g., alarmins such as S100, fibronectin fragments, hyaluronic acid fragments, collagen fragments, proteoglycan fragments, high-mobility group box, etc.), and a combination thereof. In some embodiments, markers of joint tissue dysfunction and/or degeneration may include other markers known to one of skill in the art (e.g., as described in Wei, et al., 2012, “Activation of Indian hedgehog promotes chondrocyte hypertrophy and upregulation of MMP-13 in human osteoarthritic cartilage,” Osteoarthr. Cartril. 20: 755-763 and/or Martel-Pelletier, et al., 2016, “Osteoarthritis,” Nature Reviews 2: 1-18, each of which is incorporated by reference in its entirety herein).

Accordingly, a decrease in a marker of joint tissue dysfunction and/or degeneration may include, in some embodiments, increased GAG staining, increased cartilage thickness, decreased fibrillation, increased cartilage surface smoothness, increased bone tissue density, decreased OARSI score, or a combination thereof. In some embodiments, a decrease in a marker of joint tissue dysfunction and/or degeneration may include increased levels of sGAG, increased cell proliferation (e.g., as measured by an increase in cells expressing Ki67), decreased pain and/or pain-related behaviors in the subject, decreased expression of Ihh protein, decreased expression of catabolic genes (e.g., MMP-13, MMP-3, MMP-L, aggrecanase, ADAMTS4, ADAMTS5, cathepsins, etc.), increased expression of anabolic chondrocyte genes (e.g., Sox9, collagen II, Acan, etc.), increased expression of genes involved in mitochondrial function and metabolism (e.g., Cox5a, Ndufa9), decreased expression of genes involved in mitochondrial dysfunction and/or osteoarthritis (e.g., Htra1, Dcn), increased mitochondrial biogenesis, increased mitochondria levels, decreased levels of type X collagen, decreased chondrocyte size, decreased chondrocyte sphericity, decreased levels of cytokines and/or chemokines (e.g., IL1, IL6, IL15, oncostatin M, tumor necrosis factor, CCL7, CXCL10, CCL4, VEGF, IL27, IL2, GMCSF, CCL5, etc.), decreased levels of inflammatory mediators (e.g., nitric oxide, reactive oxygen species, complement activation, etc.), decreased levels of cell-derived and/or matrix-derived products (e.g., alarmins such as S100, fibronectin fragments, hyaluronic acid fragments, collagen fragments, proteoglycan fragments, high-mobility group box, etc.), and a combination thereof.

In some embodiments, a decrease in a marker of joint tissue dysfunction and/or degeneration may include increased or enhanced joint tissue functions as determined by mechanical methods (e.g., bulk tissue specimen mechanical testing, microbeam mechanical testing, microindentation, or nanoindentation); imaging methods (e.g., computerized tomography (CT), magnetic resonance imaging (MRI), nuclear magnetic resonance (NMR), fourier-transform infrared spectroscopy (FTIR). Raman imaging, or scanning electron microscopy); chemical or physical methods (gravimetric analysis or chemical analysis of collagen crosslinks): densitometry analysis: or a combination thereof. Joint tissue structure and/or function may be improved (e.g., by any method disclosed herein) by at least about 10% (e.g., at least about 15%, at least about 20%, at least about 25%, at least about 30%, at least about 40%, at least about 50/o, at least about 60%, at least about 70%, or greater) relative to the joint tissue prior to the treatment (e.g., with the 15-PGDH inhibitor) or relative to the level present in a healthy joint tissue from the same subject. In some embodiments, the joint tissue of the subject displays at least one marker of dysfunction and/or degeneration and the joint tissue structure and/or function may be improved (e.g., after treatment with a 15-PGDH inhibitor. e.g., according to methods provided herein) to a level substantially similar to a level present in a joint tissue of a subject not displaying the at least one marker of dysfunction and/or degeneration. Joint tissue structure and/or function may be improved (e.g., by any method disclosed herein) to a level within about 10% to about 200% of a level present in a joint tissue of a subject not displaying the at least one marker of dysfunction and/or degeneration (e.g., about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200% or more). In some embodiments, joint tissue structure and/or function is improved (e.g., by any method disclosed herein) to a level within about 50% of a level present in a joint tissue of a subject not displaying the at least one marker of dysfunction and/or degeneration.

In some embodiments, joint tissue function and activity improvement is demonstrated by improvement in pain, disease activity or disability by about 50%, by about 60%, by about 70%, by about 80%, by about 90%, by about 95%, by about 100% or more. In some embodiments, joint tissue function and activity improvement is demonstrated by improvement in ACR (American College of Rheumatology) criteria, including ACR20, ACR50 or ACR70 criteria. ACR criteria is used to assess and establish improvement in tender or swollen joint counts, along with improvement in three of the following five parameters: 1) Acute phase reactant—amount of inflammation in joints as determined by C-reactive protein or sedimentation rate; 2) patient assessment—progress and response by patient to joint treatment: 3) healthcare provider assessment—progress and response assessment to treatment by healthcare provider; 4) pain—level of joint pain in patient; and 5) disability/functional questionnaire—level of interference by joint disease in daily activities.

In some embodiments, joint tissue function and activity improvement is demonstrated by decreased pain and/or pain-related behaviors. In some embodiments, pain (e.g., OA-related pain) in subjects is assessed using the Western Ontario and McMaster Universities Osteoarthritis Index (WOMAC), which is a standardized set of questionnaires used as patient-reported outcomes (PROs) by health professionals to evaluate the pain, stiffness, and physical functioning of subject joints (see, e.g., Bellamy, 1989, Seminars in Arthritis and Rheumatism, 18(4) supp. 2: 14-17, Peat et al., 2001, Ann. Rheum. Dis. 60:91e7, and Neogi, 2013, Osteoarthritis and Cartilage 21:1145-1153). In some embodiments, pain and/or pain-related behaviors can be assessed (e.g., in small animals) through gait analyses (e.g., using catwalk video based systems) and/or by testing for mechanical allodynia (e.g., using von Frey filament assay) (see, e.g., Malfait et al., 2013, Osteoarthritis and Cartilage 21:1316-1326).

Techniques for assessment of joint tissue structure and/or function are known to those skilled in the art. For example, histologic examination of cartilage proteoglycan (GAG) staining with safranin O can reveal dysfunction and/or degeneration via decreased GAG staining. Other examples include, but are not limited to, examination of fibrillation levels, examination of cartilage thickness, examination of level of cartilage surface smoothness, examination of bone tissue density, OARSI scoring, histologic examination of joint tissue for presence of additional markers described above (e.g., sGAG, type X collagen, chondrocyte size, chondrocyte sphericity, cytokines and/or chemokines, inflammatory mediators, and/or cell-derived and/or matrix derived products), measurement of cell proliferation (e.g., via measurement of cell proliferation markers such as Ki67), quantification of expression of marker genes listed above (e.g., at the protein level by techniques such as western blot, immunofluorescence, flow cytometry, or mass spectrometry; or at the mRNA level by techniques such as quantitative reverse transcriptase PCR or RNA sequencing), or combinations thereof. In some embodiments, decreased expression of anabolic chondrocyte genes (e.g., Sox9, collagen II, or Acan) indicates joint dysfunction and/or degeneration. In some embodiments, increased expression of catabolic genes (e.g., MMP-13 or MMP-3) indicates joint dysfunction and/or degeneration. In some embodiments, decreased expression of genes involved in mitochondrial function and metabolism (e.g., Cox5a, Ndufa9) indicates joint dysfunction and/or degeneration. In some embodiments, increased expression of genes involved in mitochondrial dysfunction and/or osteoarthritis (e.g., Htra1, Den) indicates joint dysfunction and/or degeneration. In some embodiments, increased pain (e.g., assessed according to any of the methods described herein) indicates joint dysfunction and/or degeneration. Joint tissue function may also be evaluated via medical techniques including examination for gross abnormalities, observation of joint movement, joint palpation, and combinations thereof.

In some embodiments, treatment (e.g., with a 15-PGDH inhibitor, e.g., according to methods provided herein) results in a reduction, decrease, attenuation, or inhibition of joint tissue dysfunction. Joint tissue dysfunction includes any characteristic that impairs the normal function of a joint, including, but not limited to, joint pain, reduced range of joint motion, soreness, and combinations thereof. The level of joint tissue dysfunction can be measured using any of the techniques described herein, or any other method known to those skilled in the art. In some embodiments, efficacy can be measured using standard clinical measurements of joint tissue function and activity, including improvement in pain, disease activity and disability (for example, Disability Index of the Health Assessment Questionnaire (HAQ); and doctor global assessment of disease activity). In some embodiments, efficacy can be measured using ACR (American College of Rheumatology) criteria, including ACR20. ACR50 or ACR80. See. e.g., van de Putte et al., Ann. Rheum. Dis 62:1168-1177 (2003). In some embodiments, treatment (e.g., with a 15-PGDH inhibitor, e.g., according to methods provided herein) results in increased expression of anabolic chondrocyte genes (e.g., Sox9, collagen II, or Acan). In some embodiments, treatment (e.g., with a 15-PGDH inhibitor, e.g., according to methods provided herein) results in decreased expression of catabolic genes (e.g., MMP-13 or MMP-3). In some embodiments, treatment (e.g., with a 15-PGDH inhibitor, e.g., according to methods provided herein) results in increased hematopoietic function and/or rejuvenation of the hematopoietic stem cell (HSC) niche.

In some embodiments, treatment (e.g., with a 15-PGDH inhibitor, e.g., according to methods provided herein) results in a reduction, decrease, attenuation, cessation, or inhibition of joint tissue degeneration and/or pain. Joint tissue degeneration includes loss, weakening, degradation, or impairment of any tissue involved in joint function, including, but not limited to, cartilage, synovium, bone, bone marrow, ligament, tendon, bursa, meniscus, and combinations thereof. In some embodiments, the joint tissue is from any joint of a subject (e.g., knee, ankle, hip, hand, wrist, elbow, spinal column, neck, shoulder, etc.). The level of joint tissue degeneration can be measured using any of the techniques described herein, or any other method known to those skilled in the art.

In some embodiments, the 15-PGDH inhibitor reduces joint tissue dysfunction and/or degeneration by at least about 10% (e.g., at least about 15%, at least about 20%, at least about 25%, at least about 30%, at least about 40%, at least about 50%, at least about 60%, at least about 70%, or greater) relative to the cells and/or tissues prior to the treatment (e.g., with the 15-PGDH inhibitor) or relative to the level present in the cells and/or tissues of a healthy joint from the same subject. In some embodiments, the subject displays at least one marker of dysfunction and/or degeneration and the 15-PGDH inhibitor reduces the at least one marker of joint tissue dysfunction and/or degeneration (e.g., after treatment with a 15-PGDH inhibitor, e.g., according to methods provided herein) to a level substantially similar to a level present in a joint tissue of a subject not displaying the at least one marker of dysfunction and/or degeneration. The at least one marker of joint tissue dysfunction and/or degeneration may be reduced (e.g., by any method disclosed herein) to a level within about 10% to about 200% or less of a level present in a joint tissue of a subject not displaying the at least one marker of dysfunction and/or degeneration (e.g., within about 40%, within about 35%, within about 30%, within about 25%, within about 20%, within about 15%, within about 10%, within about 5%, or within about 1%).

The present disclosure also provides methods of treating diseases, disorders, or conditions affecting a joint tissue, e.g., any type of arthritis, osteoarthritis, osteoporosis, rheumatoid arthritis, juvenile idiopathic arthritis, gout, systemic lupus erythematosus, seronegative spondyloarthropathy, degenerative disc disease, congenital cartilage disorders, bone disorders, and combinations thereof. In some embodiments, the disease, disorder, or condition is rheumatoid arthritis, psoriatic arthritis, juvenile idiopathic arthritis, ankylosing spondylitis, connective tissue-disease psoriasis, polyarthritis, or osteoarthritis. In some embodiments, the methods comprise administering a therapeutically effective amount of a pharmaceutical composition comprising a 15-PGDH inhibitor (e.g., as described herein) to a subject having a disease, disorder, or condition affecting a joint tissue. In some embodiments, the subject has rheumatoid arthritis, psoriatic arthritis, juvenile idiopathic arthritis, ankylosing spondylitis, connective tissue-disease psoriasis, polyarthritis, or ostcoarthritis.

The present disclosure also provides methods of measuring 15-PGDH levels in joint tissues of a subject with at least one marker of dysfunction and/or degeneration. Such methods are useful, e.g., for the use of 15-PGDH as a biomarker of joint tissue dysfunction and/or degeneration, e.g., wherein an elevated level of 15-PDGH levels or activity, e.g., an increase of 10%, 20%, 30%, 40%, 50%, 60%, 70%, 80%, 90%, 100% or more relative to a control level in a control subject without joint tissue dysfunction and/or degeneration or relative to a healthy joint in the subject is indicative of joint tissue dysfunction and/or degeneration. In such methods, 15-PGDH can be assessed in any of a number of ways, e.g., by detecting levels of a transcript encoding a 15-PGDH protein, by detecting levels of a 15-PGDH polypeptide, or by detecting 15-PGDH enzymatic activity.

In particular embodiments, the inhibition of 15-PGDH in the subject leads to an increase in PGE2 and/or PGD2, e.g., an elevation, increase, or restoration of PGE2 and/or PGD2 levels, in the joint tissue of the subject, and a decrease in PGE2 and/or PGD2 metabolites such as 15-keto-PGE2, 13,14-dihydro-15-keto-PGE2 (PGEM), 15-keto-PGD2, and 13,14-dihydro-15-keto-PGD2. In some embodiments, the inhibition also leads to increased signaling through PGE2 receptors, e.g., EP1, EP2, EP3, and/or EP4 (also known as Ptger1, Ptger2, Ptger3, Ptger4) in the joint tissue. In some embodiments, the inhibition also leads to increased signaling through PGD2 receptors, e.g., DP1 and/or DP2 (also known as PTGDR1, PTGDR2/CRTH2).

In particular embodiments, the herein-described benefits of 15-PGDH inhibitor administration in the joint tissue, e.g., improved joint tissue function, etc., occur independently of any regeneration of the joint tissue in the subject. In other words, while there may be regeneration of the joint tissue in the subject. e.g., if the joint tissue has been injured or damaged, the herein-described effects do not require the regeneration and would occur even without the regeneration. In particular embodiments, the joint tissue is not injured or damaged and has not or does not undergo regeneration.

### Subjects

The subject can be any subject, e.g., a human or other mammal, with at least one marker of joint tissue dysfunction and/or degeneration or at risk of having at least one marker of joint tissue dysfunction and/or degeneration. In some embodiments, the subject is a human. In some embodiments, the subject is an adult. In some embodiments, the subject is a child. In some embodiments, the subject is female (e.g., an adult female). In some embodiments, the subject is male (e.g., an adult male).

In some embodiments, the subject is human, and the method further comprises a step in which the human is selected for treatment with the 15-PGDH inhibitor based on a determination that the human has at least one marker of joint tissue dysfunction and/or degeneration, or on the potential for or risk of developing at least one marker of joint tissue dysfunction and/or degeneration. In some such embodiments, the human is selected based on his or her age. For example, a human can be selected for treatment based on age who is over 20, 25, 30, 35, 40, 45, 50, 55, 60, 65, 70, 75, 80, 85, 90, 95, or 100 years old or older, or any age in which the human has or potentially has at least one marker of joint tissue dysfunction and/or degeneration. In some embodiments, the subject is less than 30 years of age. In some embodiments, the subject is at least 30 years of age. In some embodiments, the human is selected based on a potential for at least one marker of joint tissue dysfunction and/or degeneration, based on the presence or potential presence of an environmental, lifestyle, or medical factor linked to joint tissue dysfunction and/or degeneration, such as family history of joint issues, diet, lack of physical activity, insufficient sleep, drug use, smoking, drinking, exposure to extreme temperatures, stress, excess weight, or health-related factors such as infections, disease, disorders, conditions, etc.

In some embodiments, the subject is determined to have joint tissue dysfunction and/or degeneration as determined using any method of assessing any measure of the function, performance, health, strength, endurance, physiological activity, or any other property of a joint tissue, e.g., a performance-based, imaging-based, physiological, molecular, cellular, or functional assay. In some embodiments, the subject is selected for treatment based on an assessment of joint tissue structure and/or function. In some embodiments, the subject is selected for treatment based on a detection of elevated levels of 15-PGDH transcript, protein, or enzymatic activity in a joint tissue, or on a detection of decreased levels of PGE2 and/or PGD2 in the joint tissue.

In some embodiments, the methods comprise an additional step subsequent to the administration of a 15-PGDH inhibitor, comprising assessing the health, function, performance, or any other property of a joint tissue in the subject, or comprising assessing the level of 15-PGDH (e.g., of 15-PGDH protein, transcript, or activity) and/or PGE2 and/or PGD2 in the joint tissue in the subject, e.g., to ascertain the potential effects of the prior administration of the 15-PGDH inhibitor on the joint tissue. In some such embodiments, the health, function, performance, 15-PGDH level, PGE2 level, PGD2 level, or other property of the joint tissue is detected or examined and compared to the health, function, performance, 15-PGDH level, PGE2 level, PGD2 level, or other property of the joint tissue prior to the administration of the 15-PGDH inhibitor or to a control value, wherein a determination that the health, function, or performance of the joint tissue has improved, that the 15-PGDH level has decreased, that the PGE2 level and/or PGD2 level has increased, in the joint tissue subsequent to the administration of the inhibitor as compared to the value obtained prior to the administration of the 15-PGDH inhibitor or relative to a control value, indicates that the 15-PGDH inhibitor has had a beneficial effect in the joint tissue of the subject.

In some embodiments, the subject is of an advanced age, e.g., 20, 25, 30, 35, 40, 45, 50, 55, 60, 65, 70, 75, 80, 85, 90, 95, or 100 years old or older. In some embodiments, the subject of an advanced age displays at least one marker of joint tissue dysfunction and/or degeneration as a result of the advanced age.

In some embodiments, the subject has sustained an injury affecting a joint tissue, e.g., cartilage injury, joint injury, trauma, anterior cruciate ligament (ACL) tear, meniscus tear, hip labral tear, rotator cuff injury, spondylosis, spinal fractures, hip fractures, degenerative spondylolisthesis, skipped disc, herniated disc, and combinations thereof. In some embodiments, the joint tissue is from any joint of a subject (e.g., knee, ankle, hip, hand, wrist, elbow, spinal column, neck, shoulder, etc.). In some embodiments, the injured subject displays at least one marker of joint tissue dysfunction and/or degeneration as a result of the injury.

In some embodiments, the subject has a disease, disorder, or condition affecting a joint tissue, e.g., any type of arthritis, osteoarthritis, osteoporosis, rheumatoid arthritis, juvenile idiopathic arthritis, gout, systemic lupus erythematosus, seronegative spondyloarthropathy, degenerative disc disease, congenital cartilage disorders, bone disorders, and combinations thereof. In some embodiments, the subject has osteoarthritis. In some embodiments, the joint tissue is from any joint of a subject (e.g., knee, ankle, hip, hand, wrist, elbow, spinal column, neck, shoulder, etc.). In some embodiments, the subject having a disease, disorder, or condition displays at least one marker of joint tissue dysfunction and/or degeneration as a result of the disease, disorder, or condition.

The present methods can be used to treat any joint tissue, or cells within such tissues, including, but not limited to, cartilage, synovium, bone, bone marrow, ligament, tendon, bursa, meniscus, and combinations thereof. In some embodiments, the joint tissue is from any joint of a subject (e.g., knee, ankle, hip, hand, wrist, elbow, spinal column, neck, shoulder, etc.).

### 5. Assessing 15-PGDH Levels

Any of a number of methods can be used to assess the level of 15-PGDH in a tissue, e.g., when using 15-PGDH as a biomarker or when assessing the efficacy of an inhibitor of 15-PGDH. For example, the level of 15-PGDH can be assessed by examining the transcription of a gene encoding 15-PGDH (e.g., the Hpgd gene), by examining the levels of 15-PGDH protein in the tissue, or by measuring the 15-PGDH enzyme activity in the tissue. Such methods can be performed on the overall tissue or on a subset of cells within the tissue.

In some embodiments, the methods involve the measurement of 15-PGDH enzyme activity, e.g., using standard methods such as incubating a candidate compound in the presence of 15-PGDH enzyme, NAD(+), and PGE2 and/or PGD2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., EP 2838533 B1.

In some embodiments, the methods involve the detection of 15-PGDH-encoding polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406: Thellin et al., Biotechnology Advances, 2009, 27:323-333; and Zheng et al, Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques. 2005, 39:75-75. Quantitative PCR and RT-PCR assays for measuring gene expression are also commercially available (e.g., TaqMan® Gene Expression Assays, ThermoFisher Scientific).

In some embodiments, the methods involve the detection of 15-PGDH protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R Twyman, ed., Garland Science, 2013.

In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA): capillary electrophoresis immunoassays (CEIA): radioimmunoassays (RIA): immunoradiometric assays (IRMA): immunofluorescence (IF); fluorescence polarization immunoassays (FPIA): and chemiluminescence assays (CL). If desired, such immunoassays can be automated. Immunoassays can also be used in conjunction with laser induced fluorescence (see. e.g., Schmalzing et al., Electrophoresis, 18:2184-93 (1997); Bao. J. Chromatogr. B. Biomed. Sci., 699:463-80 (1997)).

### 6. 15-PGDH as a Biomarker

In some embodiments, 15-PGDH may be used as a biomarker for joint tissue dysfunction and/or degeneration, or for the presence or potential for a disease, disorder, or condition affecting joint tissue. For example, a detection of an increase in 15-PGDH levels in a joint tissue, e.g., in the overall tissue or in specific cells within the joint tissue, may be indicative of joint tissue dysfunction and/or degeneration in the joint tissue, of a loss or decrease of function or health of the joint tissue related to aging, injury, disease, or disorder, or of the presence or occurrence of aging, injury, disease, disorder, or condition. For example, a detected increase of about 10%, about 20%, about 30%, about 40%, about 50%, about 60%, about 70%, about 80%, about 90%, about 100%, or more 15-PGDH in a joint tissue as compared to in a control tissue from a subject without a disease, disorder, or condition affecting joint tissue may be indicative of joint tissue dysfunction and/or degeneration in the joint tissue, of a loss or decrease of function or health of the joint tissue related to aging, injury, disease, disorder, or condition or of the presence or occurrence of aging, injury, disease, disorder, or condition.

### 7. 15-PGDH Inhibitors

Any agent that reduces, decreases, counteracts, attenuates, inhibits, blocks, downregulates, or eliminates in any way the expression, stability or activity, e.g., enzymatic activity, of 15-PGDH can be used in the present methods. Inhibitors can be small molecule compounds, peptides, polypeptides, nucleic acids, antibodies, e.g., blocking antibodies or nanobodies, or any other molecule that reduces, decreases, counteracts, attenuates, inhibits, blocks, downregulates, or eliminates in any way the expression, stability, and/or activity of 15-PGDH, e.g., the enzymatic activity of 15-PGDH.

In some embodiments, the 15-PGDH inhibitor decreases the activity, stability, or expression of 15-PGDH by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or more relative to a control level, e.g., in the absence of the inhibitor, in vivo or in vitro.

The efficacy of inhibitors can be assessed, e.g., by measuring 15-PGDH enzyme activity, e.g., using standard methods such as incubating a candidate compound in the presence of 15-PGDH enzyme, NAD(+), and PGE2 in an appropriate reaction buffer, and monitoring the generation of NADH (see, e.g., Zhang et al, (2015) Science 348: 1224), or by using any of a number of available kits such as the fluorometric PicoProbe 15-PGDH Activity Assay Kit (BioVision), or by using any of the methods and/or indices described in, e.g., EP 2838533 B1.

The efficacy of inhibitors can also be assessed, e.g., by detection of decreased polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406: Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al. BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the 15-PGDH inhibitor is considered effective if the level of expression of a 15-PGDH-encoding polynucleotide is decreased by at least about 10%, at least about 20%, at least about 30%, at least about 40%, at least about 50%, at least about 60%, at least about 70%, at least about 80%, at least about 90% or more as compared to the reference value, e.g., the value in the absence of the inhibitor, in vitro or in vivo. In some embodiments, a 15-PGDH inhibitor is considered effective if the level of expression of a 15-PGDH-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

The effectiveness of a 15-PGDH inhibitor can also be assessed by detecting protein expression or stability. e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013.

In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA): capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

For determining whether 15-PGDH protein levels are decreased in the presence of a 15-PGDH inhibitor, the method comprises comparing the level of the protein (e.g., 15-PGDH protein) in the presence of the inhibitor to a reference value, e.g., the level in the absence of the inhibitor. In some embodiments, a 15-PGDH protein is decreased in the presence of an inhibitor if the level of the 15-PGDH protein is decreased by at least about 10%, at least about 20%, at least about 30%, at least about 40%, at least about 50%, at least about 60%, at least about 70%, at least about 80%, at least about 90% or more as compared to the reference value. In some embodiments, a 15-PGDH protein is decreased in the presence of an inhibitor if the level of the 15-PGDH protein is decreased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold or more as compared to the reference value.

### Small Molecules

In particular embodiments, 15-PGDH is inhibited by the administration of a small molecule inhibitor. Any small molecule inhibitor can be used that reduces. e.g., by about 10%, about 15%, about 20%, about 25%, about 30%, about 35%, about 40%, about 45%, about 50%, about 55%, about 60%, about 65%, about 70%, about 75%, about 80%, about 85%, about 90%, or more, the expression, stability, or activity of 15-PGDH relative to a control, e.g., the expression, stability, or activity in the absence of the inhibitor. In particular embodiments, small molecule inhibitors may be used that can reduce the enzymatic activity of 15-PGDH in vitro or in vivo. Non-limiting examples of small molecule compounds that can be used in the present methods include the small molecules disclosed in EP 2838533 B1, the entire disclosure of which is herein incorporated by reference. Small molecules can include, inter alia, the small molecules disclosed in Table 2 of EP 2838533 B1, i.e., SW033291, SW033291 isomer B, SW033291 isomer A, SW033292, 413423, 980653, 405320, SW208078, SW208079, SW033290, SW208080, SW208081, SW206976. SW206977, SW206978, SW206979. SW206980, SW206992, SW208064, SW208065, SW208066, SW208067, SW208068, SW208069, SW208070, as well as combinations, derivatives, isomers, or tautomers thereof. In particular embodiments, the 15-PGDH inhibitor used is SW033291 (2-(butylsulfinyl)-4-phenyl-6-(thiophen-2-yl)thieno[2,3-b]pyridin-3-amine; PubChem CID: 3337839).

In some embodiments, the 15-PGDH inhibitor is a thiazolidinedione derivative (e.g., benzylidenethiazolidine-2,4-dione derivative) such as (5-(4-(2-(thiophen-2-yl)ethoxy)benzylidene)thiazolidine-2,4-dione), 5-(3-chloro-4-phenylethoxybenzylidene)thiazolidine-2,4-dione, 5-(4-(2-cyclohexylethoxy)benzylidene)thiazolidine-2,4-dione, 5-(3-chloro-4-(2-cyclohexylethoxy)benzyl)thiazolidine-2,4-dione, (Z)—N-benzyl-4-((2,4-dioxothiazolidin-5-ylidene)methyl)benzamide, or any of the compounds disclosed in Choi et al. (2013) Bioorganic & Medicinal Chemistry 21:44774484; Wu et al. (2010) Bioorg. Med. Chem. 18(2010) 1428-1433: Wu et al. (2011) J. Med. Chem. 54:5260-5264; or Yu et al. (2019) Biotechnology and Bioprocess Engineering 24:464475, the entire disclosures of which are herein incorporated by reference. In some embodiments, the 15-PGDH inhibitor is a COX inhibitor or chemopreventive agent such as ciglitazone (CID: 2750), or any of the compounds disclosed in Cho et al. (2002) Prostaglandins. Leukotrienes and Essential Fatty Acids 67(6):461-465, the entire disclosure of which is herein incorporated by reference.

In some embodiments, the 15-PGDH inhibitor is a compound containing a benzimidazole group, such as (1-(4-methoxyphenyl)-1H-benzo[d]imidazol-5-yl)(piperidin-1-yl)methanone (CID: 3474778), or a compound containing a triazole group, such as 3-(2,5-dimethyl-1-(p-tolyl)-1H-pyrrol-3-yl)-6,7,8,9-tetrahydro-5H-[1,2,4]triazolo[4,3-a]azepine (CID: 71307851), or any of the compounds disclosed in Duveau et al. (2015) (“Discovery of two small molecule inhibitors, ML387 and ML388, of human NAD+-dependent 15-hydroxyprostaglandin dehydrogenase,” published in Probe Reports from the NIH Molecular Libraries Program [Internet]), the entire disclosure of which is herein incorporated by reference. In some embodiments, the 15-PGDH inhibitor is 1-(3-methylphenyl)-1H-benzimidazol-5-yl)(piperidin-1-yl)methanone (CID: 4249877) or any of the compounds disclosed in Niesen et al. (2010) PLoS ONE 5(11):e13719, the entire disclosure of which is herein incorporated by reference. In some embodiments, the 15-PGDH inhibitor is 2-((6-bromo-4H-imidazo[4,5-b]pyridin-2-ylthio)methyl)benzonitrile (CID: 3245059), piperidin-1-yl(1-m-tolyl-1H-benzo[d]imidazol-5-yl)methanone (CID: 3243760), or 3-(2,5-dimethyl-1-phenyl-1H-pyrrol-3-yl)-6,7,8,9-tetrahydro-5H-[1,2,4]triazolo[4,3-a]azepine (CID: 2331284), or any of the compounds disclosed in Jadhav et al. (2011) (“Potent and selective inhibitors of NAD+-dependent 15-hydroxyprostaglandin dehydrogenase (HPGD),” published in Probe Reports from the NIH Molecular Libraries Program [Internet]), the entire disclosure of which is herein incorporated by reference.

In some embodiments, the 15-PGDH inhibitor is TD88 or any of the compounds disclosed in Seo el al. (2015) Prostaglandins, Leukotrienes and Essential Fatty Acids 97:35-41, or Shao et al. (2015) Genes & Diseases 2(4):295-298, the entire disclosures of which are herein incorporated by reference. In some embodiments, the 15-PGDH inhibitor is EEAH (Ethanol extract of Artocarpus heterophyllus) or any of the compounds disclosed in Kama (2017) Pharmacogn Mag. 2017 January; 13(Suppl 1): S122-S126, the entire disclosure of which is herein incorporated by reference.

### Inhibitory Nucleic Acids

In some embodiments, the agent comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical) to a target sequence in a 15-PGDH polynucleotide (e.g., a portion comprising at least about 20, at least about 30, at least about 40, at least about 50, at least about 60, at least about 70, at least about 80, at least about 90, or at least about 100 contiguous nucleotides, e.g., from about 20-500, about 20-250, about 20-100, about 50-500, or about 50-250 contiguous nucleotides of a 15-PGDH-encoding polynucleotide sequence (e.g., the human HPGD gene, Gene ID: 3248, including of any of its transcript variants, e.g., as set forth in GenBank Accession Nos. NM_000860.6, NM_001145816.2, NM_001256301.1, NM_001256305.1, NM_001256306.1, NM_001256307.1, or NM_001363574.1).

In some embodiments, the methods described herein comprise treating a subject, e.g., a subject with a disease, disorder, or condition associated with joint tissue dysfunction and/or degeneration such as ostcoarthritis, using an shRNA or siRNA. A shRNA is an artificial RNA molecule with a hairpin turn that can be used to silence target gene expression via the siRNA it produces in cells. See. e.g., Fire et. al., Nature 391:806-811, 1998; Elbashir et al., Nature 411:494498, 2001; Chakraborty et al., Mol Ther Nucleic Acids 8:132-143, 2017; and Bouard et al., Br. J. Pharmacol. 157:153-165, 2009. In some embodiments, a method of treating a subject, e.g., a subject with a disease, disorder, or condition associated with joint tissue dysfunction and/or degeneration such as osteoarthritis, comprises administering to the subject a therapeutically effective amount of a modified RNA or a vector comprising a polynucleotide that encodes an shRNA or siRNA capable of hybridizing to a portion of a 15-PGDH mRNA (e.g., a portion of the human 15-PGDH-encoding polynucleotide sequence set forth in any of GenBank Accession Nos. NM 000860.6, NM 001145816.2, NM 001256301.1, NM_001256305.1, NM_001256306.1, NM_001256307.1, or NM_001363574.1). In some embodiments, the vector further comprises appropriate expression control elements known in the art, including, e.g., promoters (e.g., inducible promoters or tissue specific promoters), enhancers, and transcription terminators.

In some embodiments, the agent is a 15-PGDH-specific microRNA (miRNA or miR). A microRNA is a small non-coding RNA molecule that functions in RNA silencing and post-transcriptional regulation of gene expression, miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent may be an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a 15-PGDH mRNA (e.g., a portion of a human 15-PGDH-encoding polynucleotide sequence as set forth in any of GenBank Accession Nos. NM_000860.6, NM_001145816.2, NM_001256301.1, NM_001256305.1, NM_001256306.1, NM_001256307.1, or NM_001363574.1). In some embodiments, the oligonucleotide has a length of about 10-30 nucleotides (e.g., about 10, about 12, about 14, about 16, about 18, about 20, about 22, about 24, about 26, about 28, or about 30 nucleotides). In some embodiments, the oligonucleotide has 100% complementarity to the portion of the mRNA transcript it binds. In other embodiments, the DNA oligonucleotide has less than 100% complementarity (e.g., about 95%, about 90%, about 85%, about 80%, about 75%, or about 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies. 4^{th} edition by N. S. Templeton; Translating Gene Therapy to the Clinic: Techniques and Approaches. 1^{st} edition by J. Laurence and M. Franklin; High-Throughput RNAi Screening: Methods and Protocols (Methods in Molecular Biology) by D. O. Azorsa and S. Arora; and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see. e.g., Li et al., Nuc. Acids Res. (2006), 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Antibodies

In some embodiments, the agent is an anti-15-PGDH antibody or an antigen-binding fragment thereof. In some embodiments, the antibody is a blocking antibody (e.g., an antibody that binds to a target and directly interferes with the target's function, e.g., 15-PGDH enzyme activity). In some embodiments, the antibody is a neutralizing antibody (e.g., an antibody that binds to a target and negates the downstream cellular effects of the target). In some embodiments, the antibody binds to human 15-PGDH.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)₂, Fab′, Fab, scFv, and the like. The term “antibody or antigen-binding fragment” can also encompass multi-specific and hybrid antibodies, with dual or multiple antigen or epitope specificities.

In some embodiments, an anti-15-PGDH antibody comprises a heavy chain sequence or a portion thereof, and/or a light chain sequence or a portion thereof, of an antibody sequence disclosed herein. In some embodiments, an anti-15-PGDH antibody comprises one or more complementarity determining regions (CDRs) of an anti-15-PGDH antibody as disclosed herein. In some embodiments, an anti-15-PGDH antibody is a nanobody, or single-domain antibody (sdAb), comprising a single monomeric variable antibody domain, e.g., a single VHH domain.

For preparing an antibody that binds to 15-PGDH, many techniques known in the art can be used. See. e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see. e.g., McCafferty et al., Nature 348:552-554 (1990); Marks et al., Biotechnology 10:779-783 (1992); Lou et al., PEDS 23:311 (2010); and Chao et al., Nature Protocols 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-15-PGDH antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-15-PGDH antibody comprises one or more CDR, heavy chain, and/or light chain sequences that are humanized. For humanized antibodies, methods of making humanized antibodies are known in the art. See. e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)₂, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)₂ fragments (see. e.g., Carter et al., BioTechnology. 10:163-167 (1992)). According to another approach, F(ab′)₂ fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet™ (FortéBio, Inc., Menlo Park, CA)), and western blot analysis.

### Peptides

In some embodiments, the agent is a peptide, e.g., a peptide that binds to and/or inhibits the enzymatic activity or stability of 15-PGDH. In some embodiments, the agent is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See. e.g., Tiede et al., eLife, 2017, 6:e24903. Affimers are also commercially available, e.g., from Avacta Life Sciences.

### Vectors and Modified RNA

In some embodiments, polynucleotides providing 15-PGDH inhibiting activity, e.g., a nucleic acid inhibitor such as an siRNA or shRNA, or a polynucleotide encoding a polypeptide that inhibits 15-PGDH, are introduced into cells, e.g., tissue cells, using an appropriate vector. Examples of delivery vectors that may be used with the present disclosure are viral vectors, plasmids, exosomes, liposomes, bacterial vectors, or nanoparticles. In some embodiments, any of the herein-described 15-PGDH inhibitors, e.g., a nucleic acid inhibitor or a polynucleotide encoding a polypeptide inhibitor, are introduced into cells. e.g., tissue cells, using vectors such as viral vectors. Suitable viral vectors include but not limited to adeno-associated viruses (AAVs), adenoviruses, and lentiviruses. In some embodiments, a 15-PGDH inhibitor, e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in cells of the tissue being targeted.

In some embodiments, the nucleic acid or protein inhibitors of 15-PGDH are introduced into a subject, e.g., into the tissues of a subject, using modified RNA. Various modifications of RNA are known in the art to enhance, e.g., the translation, potency and/or stability of RNA, e.g., shRNA or mRNA encoding a 15-PGDH polypeptide inhibitor, when introduced into cells of a subject. In particular embodiments, modified mRNA (mmRNA) is used, e.g., mmRNA encoding a polypeptide inhibitor of 15-PGDH. In other embodiments, modified RNA comprising an RNA inhibitor of 15-PGDH expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′ UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/simanod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(β-amino esters) can also be used See. e.g., Youn et al. (2015) Expert Opin Biol Ther, September 2; 15(9): 1337-1348: Kaczmarek et al (2017) Genome Medicine 9:60; Gan et al. (2019) Nature comm. 10: 871; Chien et al. (2015) Cold Spring Harb Perspect Med. 2015; 5:a014035; the entire disclosures of each of which are herein incorporated by reference.

### 8. Methods of Administration

The compounds described herein can be administered locally in the subject or systemically. In some embodiments, the compounds can be administered directly (locally) to joint tissue to improve joint tissue and function including, for example, intraarticular (within a joint) or periarticular (around a joint) administration, for example, with injection or direct application of the compound (or a therapeutic composition comprising the compound) within or around a joint tissue. In some embodiments, the compounds can be administered systemically for treatment of joint tissue structure and function including, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation. In an example, the compounds are administered intramuscularly, e.g., by intramuscular injection.

In some embodiments, the compound is administered in accordance with an acute regimen. In certain instances, the compound is administered to the subject once. In other instances, the compound is administered at one time point, and administered again at a second time point. In yet other instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days, 3 days, 4 days, 5 days, 6 days, a week, 2 weeks, 3 weeks, 4 weeks, a month, or more. In other embodiments, the compound is administered continuously or chronically in accordance with a chronic regimen over a desired period of time. For instance, the compound can be administered such that the amount or level of the compound is substantially constant over a selected time period.

Administration of the compound into a subject can be accomplished by methods generally used in the art. The quantity of the compound introduced may take into consideration factors such as sex, age, weight, the types of disease, disorder, or condition, stage of the disease, disorder, or condition, and the quantity needed to produce the desired result. Generally, for administering the compound for therapeutic purposes, the cells are given at a pharmacologically effective dose. By “pharmacologically effective amount” or “pharmacologically effective dose” is an amount sufficient to produce the desired physiological effect or amount capable of achieving the desired result, particularly for treating the disease, disorder, or condition, including reducing or eliminating one or more symptoms or manifestations of the disease, disorder, or condition.

The compounds described herein may be administered locally by injection into the joint tissue being targeted, or by administration in proximity to the joint tissue being targeted.

### 9. Pharmaceutical Compositions

The pharmaceutical compositions of the compounds described herein may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions described herein (see, e.g., Remington 's Pharmaceutical Sciences, 18th Ed., Mack Publishing Co., Easton, PA (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents, for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions described herein are administered in a manner compatible with the dosage formulation, and in such amount as will be therapeutically effective. The quantity to be administered depends on a variety of factors including, e.g., the age, body weight, physical activity, and diet of the individual, the disease, disorder, or condition to be treated, and the stage or severity of the disease, disorder, or condition. In certain embodiments, the size of the dose may also be determined by the existence, nature, and extent of any adverse side effects that accompany the administration of a therapeutic agent(s) in a particular individual.

It should be understood, however, that the specific dose level and frequency of dosage for any particular patient may be varied and may depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule).

In addition, more concentrated dosage forms may be prepared, from which the more dilute unit dosage forms may then be produced. The more concentrated dosage forms thus will contain substantially more than, e.g., at least about 1, about 2, about 3, about 4, about 5, about 6, about 7, about 8, about 9, about 10, or more times the amount of the therapeutic compound.

Methods for preparing such dosage forms are known to those skilled in the art (see. e.g., Remington 's Pharmaceutical Sciences, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., Remington 's Pharmaceutical Sciences, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (e.g., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharne, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease, disorder, or condition being treated, reducing side-effects caused by induction of the immune response, etc. In some embodiments, for example, the 15-PGDH inhibitors described herein are administered together with a compound to enhance PGE2 levels and/or PGD2 levels, a compound to increase signaling through the EP1, EP2, EP3, EP4, DP1, and/or DP2 receptors, and/or any other compound aiming to enhance joint structure and/or function or the function, health, or any other desired property of the tissue being targeted.

### 10. Kits

Other embodiments of the compositions described herein are kits comprising a 15-PGDH inhibitor. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for improving structure and/or function in a joint tissue of a subject. In some embodiments, the kit comprises one or more reagents for the treatment of a disease, disorder, or condition associated with joint tissue dysfunction and/or degeneration such as osteoarthritis. In some embodiments, the kit comprises an agent that antagonizes the expression or activity of 15-PGDH. In some embodiments, the kit comprises an inhibitory nucleic acid (e.g., an antisense RNA, small interfering RNA (siRNA), microRNA (miRNA), short hairpin RNA (shRNA)), or a polynucleotide encoding a 15-PGDH inhibiting polypeptide, that inhibits or suppresses 15-PGDH mRNA or protein expression or activity, e.g., enzyme activity. In some embodiments, the kit comprises a modified RNA, e.g., a modified shRNA or siRNA, or a modified mRNA encoding a polypeptide 15-PGDH inhibitor. In some embodiments, the kit further comprises one or more plasmid, bacterial or viral vectors for expression of the inhibitory nucleic acid or polynucleotide encoding a 15-PGDH-inhibiting polypeptide. In some embodiments, the kit comprises an antisense oligonucleotide capable of hybridizing to a portion of a 15-PGDH-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a 15-PGDH protein. In some embodiments, the kit comprises a blocking peptide. In some embodiments, the kit comprises an aptamer (e.g., a peptide or nucleic acid aptamer). In some embodiments, the kit comprises an affimer. In some embodiments, the kit comprises a modified RNA. In particular embodiments, the kit comprises a small molecule inhibitor, e.g., SW033291, that binds to 15-PGDH or inhibits its enzymatic activity. In some embodiments, the kit further comprises one or more additional therapeutic agents, e.g., agents for administering in combination therapy with the agent that antagonizes the expression or activity of 15-PGDH.

In some embodiments, the kits can further comprise instructional materials containing directions (e.g., protocols) for the practice of the methods described herein (e.g., instructions for using the kit for improving structure and/or function in a joint tissue of a subject; and/or for using the kit for the treatment of a disease, disorder, or condition associated with joint tissue dysfunction and/or degeneration such as osteoarthritis). While the instructional materials typically comprise written or printed materials, they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The present disclosure will be described in greater detail by way of a specific example. The following example is offered for illustrative purposes only, and is not intended to limit the disclosure in any manner. Those of skill in the art will readily recognize a variety of noncritical parameters which can be changed or modified to yield essentially the same results.

### Example 1. Materials and Methods Used in the Examples

Mice handling. All experiments and protocols were performed in accordance with the institutional guidelines of Stanford University and Administrative Panel on Laboratory Animal Care (APLAC). Aged (24-28 mo.) mice C57BL/6 were obtained from the US National Institute on Aging (NIA) aged colony. Mice were treated for 1 month once a day by intraperitoneal injection with 5 mg/kg of SW033291 (SW) (ApexBio cat #A8709) or vehicle (10% ethanol, 5% Cremophor EL (Sigma-Aldrich cat #C5135), 85% D5W (Dextrose 5% Water). Vehicle and SW treatments were performed in 3 independent studies for aged mice. Mice that developed dermatitis throughout the treatment period or with visible tumors observed at the endpoint necropsy were excluded from the study. Young (3 mo.) wild-type C57BL/6 mice were obtained from Jackson Laboratory (n=8 mice per group). Studies were performed with male mice.

Tibia loading to induce osteoarthritis in young mice. Briefly, mice were anesthetized using 2.5% isoflurane inhalation under 4 L/min of oxygen. The right tibia was positioned vertically, with the ankle upward and the knee downward in deep flexion between loading cups. Using a material testing machine (Instron ElectroPulse E1000), axial compression was applied through the knee via the upper cup, while the lower cup was fixed and linked to the load cell. The ACL tear was confirmed by the release of compressive force during compression. Mice were allowed to progress for 6 weeks after loading (n=8 for each group). Contralateral left limb served as a non-loaded control. Mice blood serum was collected via retro-orbital bleeding to do multiplex autoantibody assay. Mice were sacrificed by cervical dislocation and knee joint were collected for assessment.

Human cartilage tissue collection and treatment. Human cartilage samples were harvested from the surgical knee tissue discards of patients (n>5, age ranging from 55-75 years) undergoing total knee arthroplasty according to approved human subjects institutional review board protocols at Stanford University. Informed, written consent was given by the patients prior to the study. Cylindrical explants (4 mm diameter, 2-4 mm thickness) were extracted from the cartilage samples using standard biopsy punches (Integra, NJ. USA). Cartilage explants were cultured in DMEM-F12 medium supplemented with 10% fetal bovine serum (FBS), 25 μg mL⁻¹ ascorbic acid, 1× glutamine, and 1× antibiotic-antimycotic solution prior to experiments. To assess the effect of 15-PGDH inhibition, explants were treated with Vehicle (DMSO) or SW (10 uM) for 1 week prior to the assessment. Fresh media was added to the explants every other day.

Histology on mouse joints and human cartilage explants. Mouse joints were fixed in 4% paraformaldehyde overnight, dehydrated in ethanol and embedded in paraffin. Sagittal Sections (5 μm) of the mouse joints were cut by the Histoserv, Inc (Germantown, MD). Cartilage histology was performed by Safranin O (Saf O) staining as previously reported (Smeriglio et al., 2020, Sci Transi. Med. 12:eaax2332). Joints were assessed through blinded grading with the OARSI scoring system. Briefly, sections of mouse joints were stained with Safranin O, and cartilage degeneration was evaluated by assigning numbers 0 to 4 for increasing depth of cartilage degeneration (OARSI scoring system). For the summit scores, five regions were summed.

After 1 week of treatment with SW, explants were fixed in 4% paraformaldehyde, washed with PBS, and cryopreserved in 30% sucrose solution overnight. Explants were then embedded in Tissue-Tek OCT medium and sectioned (approximately 10 μm thick) using a Leica Cryostat. Sections were stained with hematoxylin (Sigma) for nucleus visualization, followed by Safranin O (catalog no. 731 583, Sigma) for protcoglycans and counter-staining with fast green (catalog no. F7258, Sigma). Sections were visualized using Keyence microscope at 20× magnification. Explants from at least five different patient donors and multiple (>5) sections were used for quantitative analysis.

Luminex Assay. This assay was performed at the Human Immune Monitoring Center at Stanford University. Human 76-plex or mouse 48-plex kits (eBioscience/Affymetrix) were used according to the manufacturer's recommendations. Briefly, antibody beads and samples were added to a 96-well plate and incubated at room temperature for 2 hours, followed by overnight incubation at 4° C. with shaking at 500 to 600 rpm. Plates were then washed in a w ash buffer, and biotinylated detection antibody was added for 2 hours at room temperature with shaking. Plate was washed, and streptavidin-phycoerythrin was added After 40 min incubation and washes, reading buffer was added to the wells Plates were read using a Luminex 200 instrument with a lower bound of 50 beads per sample per cytokine. Custom assay control beads were added to all wells. The raw MFI read-outs of all samples were used and analyzed by GraphPad prism.

RNA extraction and library preparation from mouse joints for RNA seq. Knee joint microdissection was performed under a stereoscope to collect cartilage tissue by carefully eliminating any tendons, synovial membrane and meniscal residue and then shaving the cartilage tissue from the underlying bone (Smeriglio et al., 2020, Sci Transl. Med. 12:eaax2332). The isolated cartilage tissue was kept in RNA later (Ambion) and stored at −80° C. for later use. Cartilage tissue was homogenized in Trizol using a bullet blender (Next Advance), followed by RNA extraction and purification using RNeasy micro-kit (Qiagen). cDNA libraries were constructed from 10 ng total RNA using SMARTer stranded Total RNA-Seq kit v3 (Takara Bio). Libraries were sequenced to 25-30×10⁶ 150-bp paired end reads per sample on an Illumina NovaSeq platform (Novogene, CA).

Differential Gene Expression Analysis. For the RNA-seq analysis, pseudo aligner Salmon was used to quantify the expression of transcripts. Sequences were also aligned against the Mus musculus genome (mm9) using the STAR aligner. A counts matrix containing the number of counts for each gene and each sample was obtained. This matrix was analyzed by DESeq2 to calculate statistical analysis of significance of genes between samples. Up or downregulated genes, with p-value and fold change cutoff of 0.05 and 2 was used, respectively. Functional analysis of differentially expressed genes was analyzed using Ingenuity Pathway Analysis (IPA) and DAVID bioinformatic tools.

Transmission electron microscopy (TEM). Knee joints were fixed in Karnovsky's fixative composed of 2% Glutaraldehyde (EMS Cat #16000) and 4% paraformaldehyde (EMS Cat #15700) in 0.1M Sodium Cacodylate (EMS Cat #12300) pH 7.4 for 4 hours at 4° C. Joints were washed with cold PBS 3× and then decalcified in 14% EDTA solution in PBS for 3 days at 4° C. Samples were washed with PBS and placed in cold/aqueous 1% osmium tetroxide (EMS Cat #19100) on a rotator and allowed to warm to room temperature (RT) for 1 hr. Samples were washed 3× with ultrafiltered water followed by staining in 1% uranyl acetate at RT for 2 hours. Samples were then dehydrated in a series of ethanol washes for 30 minutes each at RT beginning at 50%, 70% and finally moved to 4° C. overnight. They were placed in cold 95% EtOH and allowed to warm to RT, changed to 100% 2×, then Propylene Oxide (PO) for 15 min. Samples were infiltrated with EMbed-812 resin (EMS Cat #14120) mixed 1:2, 1:1, and 2:1 with PO for 2 hrs each with leaving samples in 2:1 resin to PO overnight rotating at RT in the hood. The samples were then placed into EMbed-812 for 2 to 4 hours then placed into molds w/labels and fresh resin, orientated and placed into 65° C. oven overnight.

80 nm sections were then cut using an UC7 (Leica, Wetzlar, Germany) on formvar/Carbon coated 100 mesh Cu grids, stained for 40 seconds in 3.5% Uranyl Acetate in 50% Acetone followed by staining in Sato's Lead Citrate for 2 minutes. Sections were then observed in the JEOL JEM-1400 120 kV transmission electron microscope. Images were taken using a Gatan Orius 832 4k×4k with 9 μm pixel CMOS camera.

1,9-dimethylmethylene Blue (DMMB) and Pico-green Assays. Human OA cartilage explants (n≥3 per donor and at least 5 donors) were treated with vehicle or SW (10 μM) for 7 days. After that, explants were weighed and digested in papain digestion buffer (5 mM 1-Cysteine, 100 mM Na₂HPO₄, 5 mM EDTA, 125 μg mL⁻¹ Papain, pH 7.5) overnight at 65° C. Following digestion, the samples were centrifuged at 21,000×g for 5 min at room temperature. The supernatant was then used for DMMB and Pico-green assays for the estimation of sulfated glycosaminoglycans (sGAGs) content in the OA cartilage explants according to published protocol (Sahu et al., 2021, Adv. Healthc. Mater. 10:e20021 18). Briefly, 20 μL sample was mixed with 200 μL DMMB solution (16 mg DMMB dye dissolved in an aqueous solution of 0.03 m NaCl, 40 mm glycine, 9.5% 0.1 m acetic acid, pH 3.0) and absorbance was measured at 525 nm immediately. Chondroitin-4 sulfate was utilized as a standard. The DNA content in OA explants was measured by Pico-green assay (Quant-iT Pico-green dsDNA Assay Kit, Invitrogen) as per manufacturer's instructions using the same papain digest supernatant of the OA explants. Quantified sulfated GAG content in OA cartilage explants was normalized to the weight of corresponding explants and the DNA content.

Cell cycle marker-Ki67 immunostaining. Human OA explants treated with vehicle and SW were fixed and embedded in OCT as described in previous section, 10 μm sections on poly-1-lysine-coated glass slides were obtained using a cryostat. Immunostaining was performed using a standard protocol. Briefly, tissue sections were permeabilized with 0.25% Triton X-100 for 10 min and washed with PBS. Proteinase-K treatment was also performed for 10 mins to unmask the antigens. Following that, sections were washed and blocked with 2% goat serum for 2 hr at room temperature. After blocking, the sections were incubated with a primary antibody, anti-human Ki67 antibody (1:100, catalog no. 14-5699-82, eBioscience) overnight at 4° C. Following primary antibody incubation, the slides were washed in PBS three times and incubated with goat anti-mouse Alexa fluor 546 secondary antibody at a dilution of 1:250 for 1 hour at room temperature. The slides were then washed with PBS, incubated in Hoechst 33342 (2 μg mL⁻¹, catalog no H3570, Invitrogen) for 15 min, and finally mounted with aqueous mounting medium. The stained sections were visualized under a confocal microscope (Leica) at 63× magnification. Image analysis was performed in ImageJ.

### Example 2. Systemic 15-PGDH Inhibition by Small Molecule Treatment Regenerates Cartilage in Aged Mice

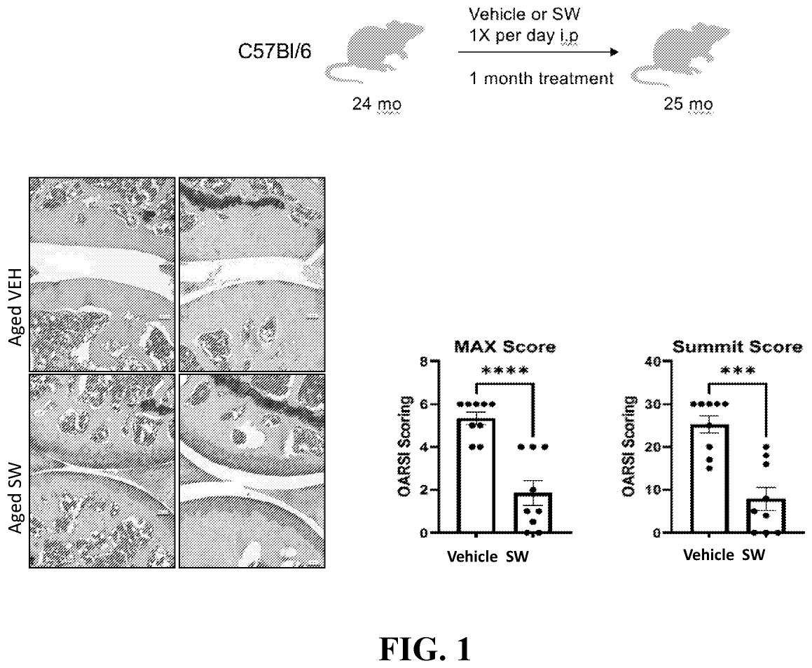

To test if the cartilage degeneration seen in aged (or osteoarthritic) joints could be overcome by systemic delivery of a small molecule inhibitor of 15-PGDH, a cohort of 24-month-old, aged mice were treated intraperitoneally with SW033291 (SW) or vehicle (FIG. 1, top panel). SW was previously extensively characterized as a specific inhibitor of 15-PGDH in vitro (ki of 0.1 nM). In vivo, SW was shown to increase PGE2 levels 2-fold, and to a lesser extent PGD2 levels, in bone marrow-, colon, lung and liver, which augmented regeneration following injury of these tissues in young mice (Zhang et al., 2015, Science 348:aaa2340). It was found that after one month of daily intraperitoneal SW treatment, 15-PGDH specific activity was significantly reduced in aged muscles, and a concomitant increase in the levels of PGE2 and PGD2 was detected by LC-MS/MS that was on par with young muscles (Palla et al., 2021, Science 371:eabc8059).

Upon testing the knee joints of these mice histologically, widespread cartilage degeneration was observed in aged mice treated with vehicle control. Representative images of safranin O staining of mice knee joints that stained the cartilage proteoglycans (GAG) showed an aging-associated decrease in GAG staining, reduction in cartilage thickness and increased fibrillation. However, after daily intraperitoneal injections of SW033291, the joints of the aged mice showed improved cartilage structure with increased thickness, GAG staining and smoother cartilage surface (FIG. 1, bottom left panel). Upon blinded scoring of mice knee joints after control or SW treatment based on the OARSI scoring system (Glasson et al., 2010, Osteoarthritis Cartilage 18 suppl. 3:S17-23), it was observed that the maximal damage score, Maximum score, as well as the cumulative Summit score, was significantly decreased for the mice knee joints after SW treatment as compared to control treated mice (FIG. 1, bottom right panel). These results in a cohort of 18 independent mice clearly showed that SW mediated inhibition of 15-PGDH was able to inhibit age-associated osteoarthritis.

### Example 3. Local 15-PGDH Inhibition in the Knee by Intra-Articular SW Injections Attenuates Post-Traumatic OA in Adult Mice

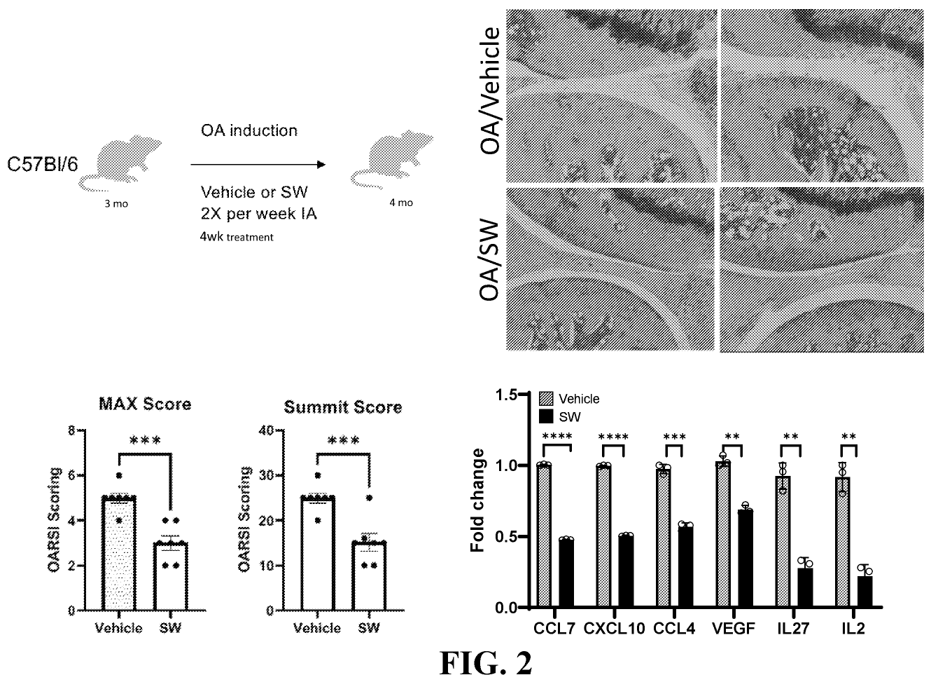

To test whether 15-PGDH inhibition would be protective in younger, adult mice upon induction of OA, OA was induced in young, adult mice (3 months old) by application of tibial compression on the mouse knee leading to anterior crucial ligament (ACL) rupture. This injury model recapitulates ACL injury frequently observed in human patients. After 1 week of injury, local intra-articular injections of vehicle or SW were given twice a week for 4 weeks. At 6 weeks, mice were euthanized, and knee joints were collected (experimental scheme shown in FIG. 2, top left panel). Safranin O-staining of mice knee joints showed reduced GAG staining and cartilage degradation in vehicle injected mice (FIG. 2, top right panel, top row), while SW injected mice showed higher GAG staining and reduced cartilage degradation (FIG. 2, top right panel, bottom row). Upon blinded scoring of mice knee joints based on the OARSI scoring system, it was observed that the maximal damage score. Maximum score, as well as the cumulative Summit score, was significantly decreased for the mice knee joints after SW injections as compared to vehicle injections (FIG. 2, bottom left panel). To assess the inflammation in these mice, serum was utilized from vehicle or SW injected mice to test for a 39-plex Luminex cytokine panel. An overall reduction of inflammatory cytokine levels was observed in SW as compared to vehicle treated mice with significant downregulation of CCL7, CXCL10, CCL4, VEGF, IL27 and IL2 (FIG. 2, bottom right panel). These observations corroborated that local 15-PGDH inhibition by SW treatment not only enhances cartilage regeneration but also decreases the inflammation in osteoarthritic mice.

### Example 4. 15-PGDH Inhibition Alters Multiple Pathways to Rejuvenate Cartilage in Aged Mice

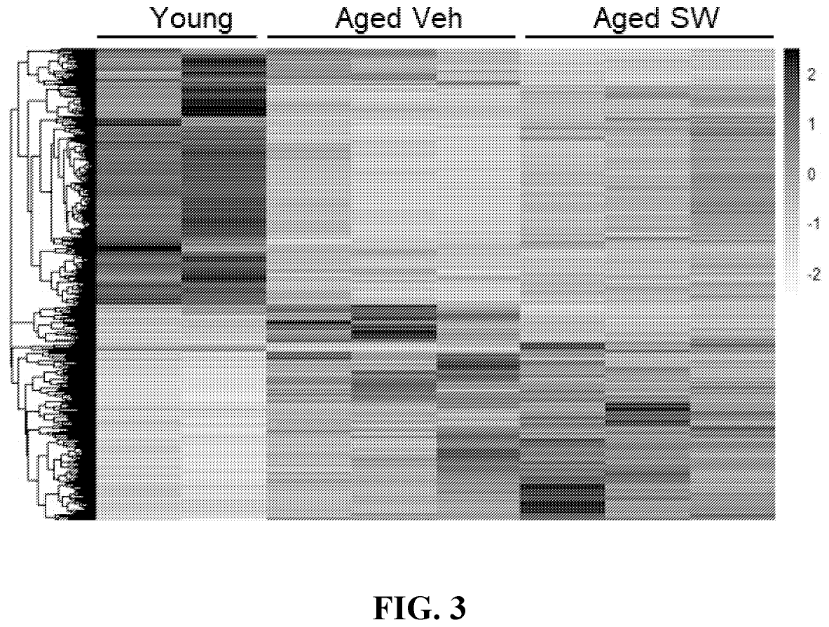

To identify the downstream molecular pathways through which 15-PGDH may exert its effect on aged cartilage, transcriptomic analysis of vehicle and SW treated cartilage from aged mice (24 mo) was performed and compared to cartilage from young adult mice (3 mo) through RNA-seq. FIG. 3 shows a heatmap of the differentially expressed genes (DEGs) among the three groups—young cartilage and vehicle or SW treated aged cartilage. Among 301 differentially expressed genes (DEGs), 147 were upregulated and 154 were downregulated in aged cartilage treated with SW compared to the vehicle. Table 1 shows Ingenuity Pathway Analysis (IPA) for significantly altered pathways in cartilage of aged mice treated systemically with vehicle or SW. Table 2 shows IPA for significantly altered pathways in cartilage of young and vehicle-treated aged mice. Table 3 shows the fold change (mean and standard deviation (“STD”)) for significantly altered genes associated with the mitochondrial oxidative phosphorylation pathway identified in Table 1 and Table 2. Table 4 shows the fold change (mean and standard deviation (“STD”)) for significantly altered genes associated with the osteoarthritis pathway identified in Table 1 and Table 2. IPA revealed significant upregulation ofgenes involved in mitochondrial function and metabolism in SW treated cartilage (Table 1). COX5a and Ndufa9, enzymes important for the functioning of mitochondria and energy production, were found to be significantly upregulated in SW treated cartilage almost to the levels observed in young cartilage (Table 3). On the contrary, pathways/genes involved in mitochondrial dysfunction and osteoarthritis were significantly upregulated in vehicle treated aged cartilage compared to the young cartilage (Table 2 and Table 4). These observations show that 15-PGDH inhibition through SW treatment affects multiple pathways to rejuvenate aged cartilage towards a younger phenotype.

TABLE 1
Enriched pathways in aged SW treated mice
versus aged vehicle treated mice.

| Ingenuity Canonical Pathways | −log(p-value) |
|---|---|
| Oxidative Phosphorylation | 6.1 |
| Mitochondrial Dysfunction | 4.44 |
| Sirtuin Signaling Pathway | 4.36 |
| Osteoarthritis Pathway | 3.3 |
| Phagosome formation | 2.434 |
| Regulation of EMT in development | 2.417 |
| Antigen Presentation Pathway | 1.987 |
| Endocytosis Signaling | 1.91 |
| FAK Signaling | 1.9 |

TABLE 2
Enriched pathways in aged vehicle treated mice versus young mice.

| Ingenuity Canonical Pathways | −log(p-value) |
|---|---|
| Mitochondrial Dysfunction | 59 |
| Oxidative Phosphorylation | 57.4 |
| Sirtuin Signaling Pathway | 39.5 |
| Estrogen Receptor Signaling | 26.5 |
| Glucocorticoid Receptor Signaling | 20.3 |
| Calcium Signaling | 16.6 |
| TCA Cycle II (Eukaryotic) | 13.7 |
| Actin Cytoskeleton Signaling | 10 |
| Osteoarthritis Pathway | 3.164 |

TABLE 3
Fold change for oxidative phosphorylation-related genes.

| | Young | | Aged vehicle treated | | Aged SW treated | |
|---|---|---|---|---|---|---|
| | Mean | STD | Mean | STD | Mean | STD |
| Cox5a | 5.72 | 0.26 | 1.30 | 0.43 | 3.42 | 0.83 |
| Ndufa9 | 3.60 | 0.66 | 1.13 | 0.13 | 2.17 | 0.56 |
| Ndufs7 | 4.50 | 0.94 | 1.23 | 0.29 | 2.63 | 0.73 |
| Atp5j | 3.04 | 0.52 | 1.18 | 0.16 | 2.12 | 0.48 |
| Atp5a1 | 3.16 | 0.47 | 1.15 | 0.23 | 2.05 | 0.36 |
| Uqcrc1 | 3.44 | 0.32 | 1.13 | 0.22 | 2.14 | 0.62 |
| Atpaf1 | 2.76 | 0.32 | 1.17 | 0.17 | 2.34 | 0.48 |
| Ndufa4 | 4.34 | 0.41 | 1.45 | 0.45 | 2.80 | 0.36 |
| mt-Nd1 | 4.77 | 0.14 | 1.27 | 0.30 | 2.42 | 0.51 |
| mt-Nd4I | 36.98 | 7.02 | 3.97 | 2.65 | 15.11 | 4.17 |

TABLE 4
Fold change for osteoarthritis-related genes.

| | Young | | Aged vehicle treated | | Aged SW treated | |
|---|---|---|---|---|---|---|
| | Mean | STD | Mean | STD | Mean | STD |
| Htra1 | 2.02 | 0.75 | 2.87 | 0.80 | 1.37 | 0.34 |
| Dcn | 1.63 | 0.08 | 2.61 | 0.65 | 1.15 | 0.16 |
| Itgb3 | 1.28 | 0.39 | 4.11 | 1.09 | 10.64 | 5.26 |
| Adamts5 | 1.32 | 0.23 | 2.36 | 0.75 | 1.05 | 0.06 |
| Smad9 | 1.32 | 0.45 | 4.81 | 0.73 | 1.45 | 0.20 |
| Itga2b | 1.02 | 0.03 | 2.61 | 0.62 | 8.42 | 4.03 |
| Gli2 | 2.24 | 1.11 | 5.00 | 1.85 | 1.34 | 0.39 |
| Creb5 | 1.30 | 0.38 | 2.21 | 0.85 | 1.02 | 0.02 |
| Tcf4 | 1.20 | 0.05 | 1.79 | 0.34 | 1.06 | 0.06 |
| Itgad | 1.85 | 1.21 | 4.27 | 1.50 | 25.59 | 5.15 |

### Example 5. 15-PGDH Inhibition Boosts Mitochondrial Biogenesis in Aged Cartilaee

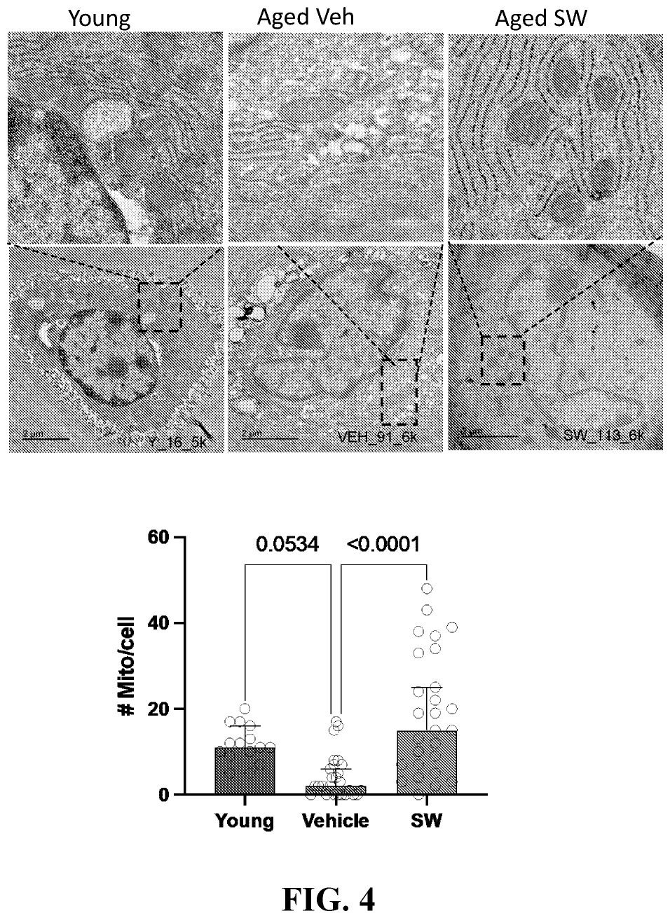

Since 15-PGDH inhibition in aged cartilage led to an upregulation of genes and pathways associated with mitochondrial biogenesis, mitochondrial biogenesis was directly investigated in young and aged chondrocytes in articular cartilage in mice knee joints. Transmission electron microscopy (TEM) images revealed that there was a significant reduction in the total number of mitochondria per cell in aged cartilage (24 month old mice) when compared to young cartilage (3 month old mice) (FIG. 4, top panel). Interestingly, a significant increase in the number of mitochondria per cell was observed in histological sections of SW treated compared to vehicle treated aged cartilage (FIG. 4, top panel). Upon quantification of mitochondria per cell using the Image j software, these changes were found to be significant with SW treatment restoring the mitochondria number in aged chondrocytes to the level of young cartilage (FIG. 4, bottom panel).

### Example 6. 15-PGDH Inhibition in Human OA Cartilage Explants Induces a Regenerative Response and Reduces Inflammation

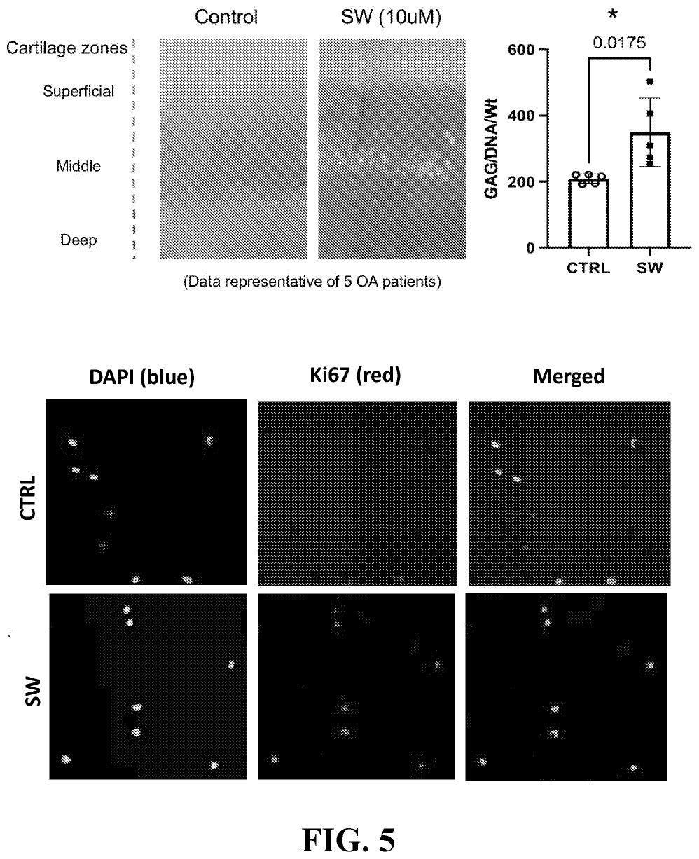

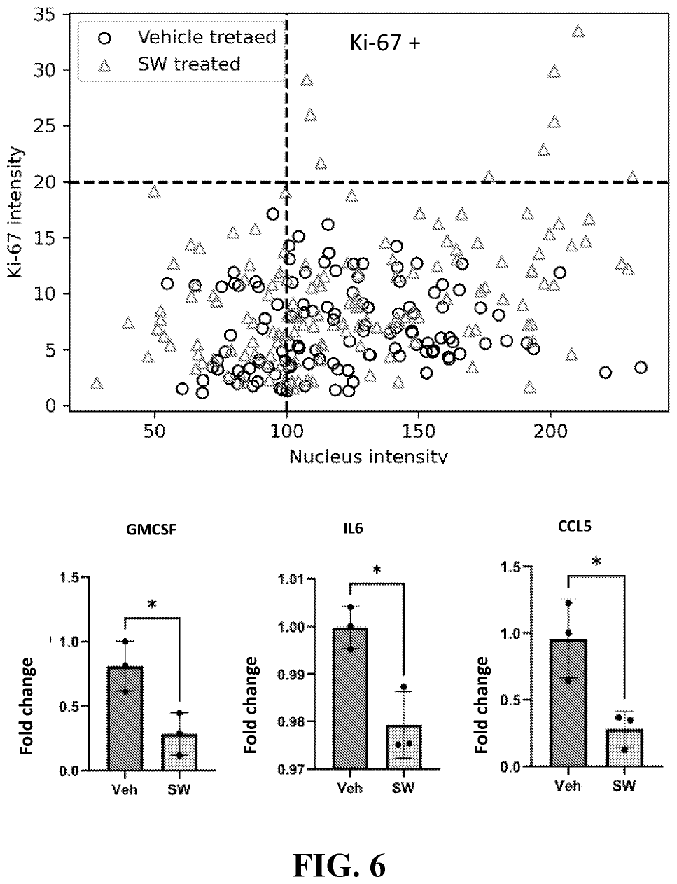

Next, the effects of 15-PGDH inhibition in human OA cartilage were tested. Human articular cartilage was isolated from the surgically discarded femoral heads from patients undergoing total knee replacement under a Stanford approved IRB protocol. Similar sized OA cartilage explants of 4 mm diameter were punched and cultured as previously described (Sahu et al., 2021, Adv. Healthc. Mater. 10:e2002118). OA explants were treated with vehicle or SW for 1 week. Proteoglycan content was assessed in the cartilage explant sections by Safranin O staining, and an increase in GAG staining was observed across all different zones of the cartilage with SW treatment especially middle and deep zone (FIG. 5, top left panel). Sulphated glycosaminoglycans (sGAG) were also independently quantified through measurements with the DMMB dye. It was observed that the GAG content measured through this biochemical assay was significantly increased in human explants treated with SW compared to vehicle (FIG. 5, top right panel). Next, the effect of SW treatment on cell proliferation was investigated. Upon immunostaining with the cell cycle marker Ki67, an increase in Ki67 positive cells in SW versus vehicle treated explants was observed (FIG. 5, lower panel). This increase was also corroborated by Ki67 intensity distribution plot that showed increased Ki67 expression in SW versus vehicle treated cells (FIG. 6, upper panel). Next, to test the effect of 15-PGDH inhibition on inflammation, a 72-plex Luminex cytokine panel was used to assess cytokine levels in the culture medium of vehicle or SW treated human OA explants. A significant reduction in the levels of GMCSF, IL6 and CCL5 was observed in the secretome of SW treated explants (FIG. 6, bottom panel), showing that like in mice, SW treatment of human OA cartilage also led to a decrease in inflammatory cytokines.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### EXEMPLARY EMBODIMENTS

Exemplary embodiments provided in accordance with the presently disclosed subject matter include, but are not limited to, the claims and the following embodiments:

Embodiment 1: a method of improving the structure and/or function of a joint tissue of a subject, the method comprising: administering to the subject an amount of a 15-hydroxyprostaglandin (15-PGDH) inhibitor effective to inhibit 15-PGDH activity and/or reduce 15-PGDH levels in the subject, thereby improving the structure and/or function of the joint tissue of the subject.
Embodiment 2: the method of embodiment 1, wherein the administering increases a level of prostaglandin E2 (PGE2) and/or prostaglandin D2 (PGD2) in the joint tissue of the subject.
Embodiment 3: the method of embodiment 2, wherein the level of PGE2 and/or PGD2 in the joint tissue is increased relative to the joint tissue prior to the administering of the 15-PGDH inhibitor.
Embodiment 4: the method of any one of embodiments 1-3, wherein the joint tissue of the subject displays at least one marker of dysfunction and/or degeneration.
Embodiment 5: the method of embodiment 4, wherein the at least one marker is selected from the group consisting of decreased GAG staining, reduction in cartilage thickness, increased fibrillation, reduction in cartilage surface smoothness, reduction in bone tissue density, increased OARSI score, decreased levels of sGAG, decreased cell proliferation, increased pain and/or pain-related behaviors in the subject, increased expression of Indian hedgehog (Ihh) protein, increased expression of catabolic genes, decreased expression of anabolic chondrocyte genes, decreased expression of genes involved in mitochondrial function and metabolism, increased expression of genes involved in mitochondrial dysfunction and/or ostcoarthritis, decreased mitochondrial biogenesis, decreased mitochondrial levels, increased levels of type X collagen, increased chondrocyte size, increased chondrocyte sphericity, increased levels of cytokines and/or chemokines, increased levels of inflammatory mediators, increased levels of cell-derived and/or matrix-derived products, and a combination thereof.
Embodiment 6: the method of embodiment 4 or 5, wherein the administering reduces the at least one marker of joint tissue dysfunction and/or degeneration.
Embodiment 7: the method of any one of embodiments 4-6, wherein the joint tissue dysfunction and/or degeneration is a result of aging.
Embodiment 8: the method of any one of embodiments 4-6, wherein the joint tissue dysfunction and/or degeneration is a result of injury.
Embodiment 9: the method of embodiment 8, wherein the injury is selected from the group consisting of cartilage injury, joint injury, trauma, anterior cruciate ligament (ACL) tear, meniscus tear, hip labral tear, rotator cuff injury, spondylosis, spinal fractures, hip fractures, degenerative spondylolisthesis, slipped disc, herniated disc, and combinations thereof.
Embodiment 10: the method of any one of embodiments 4-6, wherein the joint tissue dysfunction and/or degeneration is a result of a disease, disorder, or condition.
Embodiment 11: the method of embodiment 10, wherein the disease, disorder, or condition is selected from the group consisting of osteoarthritis, other arthritis types, osteoporosis, rheumatoid arthritis, juvenile idiopathic arthritis, gout, systemic lupus erythematosus, seronegative spondyloarthropathy, degenerative disc disease, congenital cartilage disorders, bone disorders, and combinations thereof.
Embodiment 12: the method of embodiment 10, wherein the disease, disorder, or condition is osteoarthritis.
Embodiment 13: the method of any one of embodiments 4-12, wherein a level of PGE2 and/or PGD2 in the joint tissue is increased to a level substantially similar to a level present in a joint tissue of a subject not displaying the at least one marker of dysfunction and/or degeneration.
Embodiment 14: the method of any one of embodiments 4-13, wherein a level of PGE2 and/or PGD2 in the joint tissue is increased to a level within 10% to 200% of a level present in a joint tissue of a subject not displaying the at least one marker of dysfunction and/or degeneration.
Embodiment 15: the method of any one of embodiments 1-14, wherein the joint tissue is selected from the group consisting of cartilage, synovium, bone, bone marrow, ligament, tendon, bursa, meniscus, and combinations thereof.
Embodiment 16: the method of any one of embodiments 1-15, wherein the structure and/or function of the joint tissue is improved relative to the joint tissue prior to the administering of the 15-PGDH inhibitor.
Embodiment 17: the method of any one of embodiments 1-16, wherein the method results in increased GAG staining, increased cartilage thickness, decreased fibrillation, increased cartilage surface smoothness, increased bone tissue density, decreased OARSI score, increased levels of sGAG, increased cell proliferation, decreased pain and/or pain-related behaviors in the subject, decreased expression of Indian hedgehog (Ihh) protein, decreased expression of catabolic genes, increased expression of anabolic chondrocyte genes, increased expression of genes involved in mitochondrial function and metabolism, decreased expression of genes involved in mitochondrial dysfunction and/or osteoarthritis, increased mitochondrial biogenesis, increased mitochondria levels, decreased levels of type X collagen, decreased chondrocyte size, decreased chondrocyte sphericity, decreased levels of cytokines and/or chemokines, decreased levels of inflammatory mediators, decreased levels of cell-derived and/or matrix-derived products, or any combination thereof.
Embodiment 18: the method of any one of embodiments 1-17, wherein the method results in a decreased level of a PGE2 and/or PGD2 metabolite in the joint tissue relative to the joint tissue prior to the administering of the 15-PGDH inhibitor.
Embodiment 19: the method of any one of embodiments 4-18, wherein the method results in a level of a PGE2 and/or PGD2 metabolite in the joint tissue that is substantially similar to a level present in a joint tissue of a subject not displaying the at least one marker of dysfunction and/or degeneration.
Embodiment 20: the method of embodiment 18 or 19, wherein the PGE2 and/or PGD2 metabolite is selected from the group consisting of 15-keto PGE2 and 13,14-dihydro-15-keto PGE2.
Embodiment 21: the method of any one of embodiments 1-20, wherein the 15-PGDH inhibitor is selected from the group consisting of a small molecule compound, a blocking antibody, a nanobody, and a peptide.
Embodiment 22: the method of any one of embodiments 1-21, wherein the 15-PGDH inhibitor is SW033291.
Embodiment 23: the method of any one of embodiments 1-20, wherein the 15-PGDH inhibitor is selected from the group consisting of an antisense oligonucleotide, microRNA, siRNA, and shRNA.
Embodiment 24: the method of any one of embodiments 1-23, wherein the subject is a human.
Embodiment 25: the method of any one of embodiments 1-24, wherein the subject is less than 30 years of age.
Embodiment 26: the method of any one of embodiments 1-24, wherein the subject is at least 30 years of age.
Embodiment 27: the method of any one of embodiments 1-26, wherein the 15-PGDH inhibitor reduces or blocks 15-PGDH expression.
Embodiment 28: the method of any one of embodiments 1-27, wherein the 15-PGDH inhibitor reduces or blocks enzymatic activity of 15-PGDH.

What is claimed is:

1. A method of improving the structure and/or function of a joint tissue of a subject, the method comprising: administering to the subject an amount of a 15-hydroxyprostaglandin (15-PGDH) inhibitor effective to inhibit 15-PGDH activity and/or reduce 15-PGDH levels in the subject, thereby improving the structure and/or function of the joint tissue of the subject.

2. The method of claim 1, wherein the administering increases a level of prostaglandin E2 (PGE2) and/or prostaglandin D2 (PGD2) in the joint tissue of the subject.

3. The method of claim 2, wherein the level of PGE2 and/or PGD2 in the joint tissue is increased relative to the joint tissue prior to the administering of the 15-PGDH inhibitor.

4. The method of claim 1, wherein the joint tissue of the subject displays at least one marker of dysfunction and/or degeneration.

5. The method of claim 4, wherein the at least one marker is selected from the group consisting of decreased GAG staining, reduction in cartilage thickness, increased fibrillation, reduction in cartilage surface smoothness, reduction in bone tissue density, increased OARSI score, decreased levels of sGAG, decreased cell proliferation, increased pain and/or pain-related behaviors in the subject, increased expression of Indian hedgehog (Ihh) protein, increased expression of catabolic genes, decreased expression of anabolic chondrocyte genes, decreased expression of genes involved in mitochondrial function and metabolism, increased expression of genes involved in mitochondrial dysfunction and/or osteoarthritis, decreased mitochondrial biogenesis, decreased mitochondrial levels, increased levels of type X collagen, increased chondrocyte size, increased chondrocyte sphericity, increased levels of cytokines and/or chemokines, increased levels of inflammatory mediators, increased levels of cell-derived and/or matrix-derived products, and a combination thereof.

6. The method of claim 4, wherein the administering reduces the at least one marker of joint tissue dysfunction and/or degeneration.

7. The method of claim 4, wherein the joint tissue dysfunction and/or degeneration is a result of aging.

8. The method of claim 4, wherein the joint tissue dysfunction and/or degeneration is a result of injury.

9. The method of claim 8, wherein the injury is selected from the group consisting of cartilage injury, joint injury, trauma, anterior cruciate ligament (ACL) tear, meniscus tear, hip labral tear, rotator cuff injury, spondylosis, spinal fractures, hip fractures, degenerative spondylolisthesis, slipped disc, herniated disc, and combinations thereof.

10. The method of claim 4, wherein the joint tissue dysfunction and/or degeneration is a result of a disease, disorder, or condition.

11. The method of claim 10, wherein the disease, disorder, or condition is selected from the group consisting of osteoarthritis, other arthritis types, osteoporosis, rheumatoid arthritis, juvenile idiopathic arthritis, gout, systemic lupus erythematosus, seronegative spondyloarthropathy, degenerative disc disease, congenital cartilage disorders, bone disorders, and combinations thereof.

12. The method of claim 10, wherein the disease, disorder, or condition is osteoarthritis.

13. The method of claim 4, wherein a level of PGE2 and/or PGD2 in the joint tissue is increased to a level substantially similar to a level present in a joint tissue of a subject not displaying the at least one marker of dysfunction and/or degeneration.

14. The method of claim 4, wherein a level of PGE2 and/or PGD2 in the joint tissue is increased to a level within 10% to 200% of a level present in a joint tissue of a subject not displaying the at least one marker of dysfunction and/or degeneration.

15. The method of claim 1, wherein the joint tissue is selected from the group consisting of cartilage, synovium, bone, bone marrow, ligament, tendon, bursa, meniscus, and combinations thereof.

16. The method of claim 1, wherein the structure and/or function of the joint tissue is improved relative to the joint tissue prior to the administering of the 15-PGDH inhibitor.

17. The method of claim 1, wherein the method results in increased GAG staining, increased cartilage thickness, decreased fibrillation, increased cartilage surface smoothness, increased bone tissue density, decreased OARSI score, increased levels of sGAG, increased cell proliferation, decreased pain and/or pain-related behaviors in the subject, decreased expression of Indian hedgehog (Ihh) protein, decreased expression of catabolic genes, increased expression of anabolic chondrocyte genes, increased expression of genes involved in mitochondrial function and metabolism, decreased expression of genes involved in mitochondrial dysfunction and/or osteoarthritis, increased mitochondrial biogenesis, increased mitochondria levels, decreased levels of type X collagen, decreased chondrocyte size, decreased chondrocyte sphericity, decreased levels of cytokines and/or chemokines, decreased levels of inflammatory mediators, decreased levels of cell-derived and/or matrix-derived products, or any combination thereof.

18. The method of claim 1, wherein the method results in a decreased level of a PGE2 and/or PGD2 metabolite in the joint tissue relative to the joint tissue prior to the administering of the 15-PGDH inhibitor.

19. The method of claim 18, wherein the PGE2 and/or PGD2 metabolite is selected from the group consisting of 15-keto PGE2 and 13,14-dihydro-15-keto PGE2.

20. The method of claim 4, wherein the method results in a level of a PGE2 and/or PGD2 metabolite in the joint tissue that is substantially similar to a level present in a joint tissue of a subject not displaying the at least one marker of dysfunction and/or degeneration.

21. The method of claim 20, wherein the PGE2 and/or PGD2 metabolite is selected from the group consisting of 15-keto PGE2 and 13,14-dihydro-15-keto PGE2.

22. The method of claim 1, wherein the 15-PGDH inhibitor is selected from the group consisting of a small molecule compound, a blocking antibody, a nanobody, and a peptide.

23. The method of claim 1, wherein the 15-PGDH inhibitor is SW033291.

24. The method of claim 1, wherein the 15-PGDH inhibitor is selected from the group consisting of an antisense oligonucleotide, microRNA, siRNA, and shRNA.

25. The method of claim 1, wherein the subject is a human.

26. The method of claim 1, wherein the subject is less than 30 years of age.

27. The method of claim 1, wherein the subject is at least 30 years of age.

28. The method of claim 1, wherein the 15-PGDH inhibitor reduces or blocks 15-PGDH expression.

29. The method of claim 1, wherein the 15-PGDH inhibitor reduces or blocks enzymatic activity of 15-PGDH.
